SEQUENCE LISTING (13 sequences)

SEQ ID NO: 1 - protein, 176 aa, Artificial Sequence
  Description of Artificial Sequence: Synthetic polypeptide
MKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLSLRTSTHPEST

SEQ ID NO: 2 - DNA, 20 bp, Artificial Sequence
  Description of Artificial Sequence: Synthetic primer
gaccccaaaatcagcgaaat

SEQ ID NO: 3 - DNA, 24 bp, Artificial Sequence
  Description of Artificial Sequence: Synthetic primer
tctggttactgccagttgaatctg

SEQ ID NO: 4 - DNA, 24 bp, Artificial Sequence
  Description of Artificial Sequence: Synthetic probe
accccgcattacgtttggtggacc

SEQ ID NO: 5 - DNA, 20 bp, Artificial Sequence
  Description of Artificial Sequence: Synthetic primer
ttacaaacattggccgcaaa

SEQ ID NO: 6 - DNA, 18 bp, Artificial Sequence
  Description of Artificial Sequence: Synthetic primer
gcgcgacattccgaagaa

SEQ ID NO: 7 - DNA, 23 bp, Artificial Sequence
  Description of Artificial Sequence: Synthetic probe
acaatttgcccccagcgcttcag

SEQ ID NO: 8 - DNA, 22 bp, Artificial Sequence
  Description of Artificial Sequence: Synthetic primer
gggagccttgaatacaccaaaa

SEQ ID NO: 9 - DNA, 21 bp, Artificial Sequence
  Description of Artificial Sequence: Synthetic primer
tgtagcacgattgcagcattg

SEQ ID NO: 10 - DNA, 24 bp, Artificial Sequence
  Description of Artificial Sequence: Synthetic probe
aycacattggcacccgcaatcctg

SEQ ID NO: 11 - DNA, 19 bp, Artificial Sequence
  Description of Artificial Sequence: Synthetic primer
agatttggacctgcgagcg

SEQ ID NO: 12 - DNA, 20 bp, Artificial Sequence
  Description of Artificial Sequence: Synthetic primer
gagcggctgtctccacaagt

SEQ ID NO: 13 - DNA, 23 bp, Artificial Sequence
  Description of Artificial Sequence: Synthetic probe
ttctgacctgaaggctctgcgcg

(19) United States
(12) Patent Application Publication
(10) Pub. No.: US 2023/0285510 A1
(43) Pub. Date: Sep. 14, 2023

(54) TREATMENT OF CORONAVIRUS INFECTION WITH INTERFERON LAMBDA

(71) Applicant: Eiger Biopharmaceuticals, Inc., Palo Alto, CA (US)

(72) Inventors: Jeffrey GLENN, Palo Alto, CA (US); Ingrid CHOONG, Palo Alto, CA (US); Colin HISLOP, Palo Alto, CA (US)

(73) Assignee: Eiger Biopharmaceuticals, Inc., Palo Alto, CA (US)

(21) Appl. No.: 17/760,279

(22) PCT Filed: Feb. 5, 2021
(86) PCT No.: PCT/US2021/016963

§ 371 Date: Aug. 5, 2022

Related U.S. Application Data
(60) Provisional application No. 62/971,194, filed on Feb. 6, 2020.
(60) Provisional application No. 63/017,614, filed on Apr. 29, 2020.
(60) Provisional application No. 63/021,552, filed on May 7, 2020.
(60) Provisional application No. 63/091,881, filed on Oct. 14, 2020.
(60) Provisional application No. 63/093,334, filed on Oct. 19, 2020.

(51) Int. Cl. A61K 38/21; A61P 31/14; A61K 9/00

(52) U.S. Cl. CPC A61K 38/21; A61K 9/0019; A61P 31/14

(57) ABSTRACT

Methods of treating a coronavirus infection in a human subject are provided. In some embodiments, the method comprises subcutaneously administering to the subject a therapeutically effective amount of pegylated interferon lambda-1a.

### RELATED APPLICATIONS

This application claims priority to each of U.S. Provisional Application 62/971,194, filed on Feb. 6, 2020, U.S. Provisional Application 63/017,614, filed on Apr. 29, 2020, U.S. Provisional Application 63/021,552, filed on May 7, 2020, U.S. Provisional Application 63/091,881, filed on Oct. 14, 2020, and U.S. Provisional Application 63/093,334, filed on Oct. 19, 2020, each of which are incorporated by reference in their entireties for all purposes.

### SEQUENCE LISTING

This instant application contains a Sequence Listing, which is concurrently submitted electronically in ASCII format with the specification and is hereby incorporated by reference in its entirety. Said ASCII copy, created on Feb. 1, 2021, is named 097854-1233250-SL.txt and is 4,784 bytes in size.

### FIELD

The present disclosure provides methods for treating coronavirus virus infection, including the 2019-nCoV virus infection (SARS-CoV-2), and so relates to the fields of chemistry, medicinal chemistry, medicine, molecular biology, and pharmacology.

### BACKGROUND

Coronaviruses (CoV) are a large family of viruses that cause illness ranging from the common cold to more severe diseases such as Middle East Respiratory Syndrome (MERS-CoV) and Severe Acute Respiratory Syndrome (SARS-CoV). Coronaviruses are zoonotic, meaning they are transmitted between animals and people. For example, detailed investigations found that SARS-CoV was transmitted from civet cats to humans and MERS-CoV from dromedary camels to humans. Several known coronaviruses are circulating in animals that have not yet infected humans.

A novel coronavirus (nCoV) is a new strain that has not been previously identified in humans, for example, SARS-CoV-2. SARS-CoV-2 is a novel coronavirus that has led to a global pandemic due to its relatively high transmissibility and potential to cause severe acute respiratory disease. See Huang C et al. “Clinical features of patients infected with 2019 novel coronavirus in Wuhan, China,” Lancet 02 2020; 395(10223):497-506. doi:10.1016/S0140-6736(20)30183-5. Common signs of infection include respiratory symptoms, fever, cough, shortness of breath, breathing difficulties, gastrointestinal symptoms, and covid-toes. In more severe cases, infection can cause pneumonia, severe acute respiratory syndrome, kidney failure and even death. Higher morbidity and mortality rates have been consistently observed in older human populations throughout the COVID-19 pandemic. Additionally, SARS-CoV-2 shows higher morbidity and mortality rates in individuals having cancer, chronic kidney disease, chronic obstructive pulmonary disease, Down Syndrome, heart conditions (such as heart failure, coronary artery disease, or cardiomyopathies), an immunocompromised state from solid organ transplant, obesity (including severe obesity), pregnancy, sickle cell disease, smoking, or Type 2 diabetes mellitus.

There are currently no approved therapies for treatment of COVID-19 infection in outpatients. See Cao B et al., “ATrial of Lopinavir-Ritonavir in Adults Hospitalized with Severe Covid-19,” N. Engl. J. Med. March 2020; doi:10.1056/NEJMoa2001282. Standard recommendations to prevent infection spread include regular hand washing, covering mouth and nose when coughing and sneezing, and thoroughly cooking meat and eggs. In addition, it is recommended to avoid close contact with anyone showing symptoms of respiratory illness such as coughing and sneezing.

There continues to be an ongoing need for agents to treat Coronavirus infection, including novel forms that are zoonotic and have begun to infect humans, such as SARS-CoV-2.

### BRIEF SUMMARY

Interferon lambda signals through the interferon lambda receptors that have a restricted cellular expression pattern. Interferon lambda also exhibits distinct antiviral activities from interferon alpha, due in part to the differences in expression of the interferon receptors. In one aspect, methods of treating a coronavirus infection in a human subject are provided. In some embodiments, the method comprises subcutaneously administering to the subject a therapeutically effective amount of pegylated interferon lambda-1a (lambda).

In some embodiments, the method comprises administering the pegylated interferon lambda for a first treatment period and a second treatment period. In some embodiments, the method comprises administering the pegylated interferon lambda for a first treatment period, a second treatment period, and a third treatment period. In some embodiments, the first treatment period is longer than the second treatment period. In some embodiments, the second treatment period is longer than the first treatment period. In some embodiments, the first treatment period and the second treatment period are the same length of time. In some embodiments, the first treatment period has a duration of at least 8 weeks. In some embodiments, the first treatment period has a duration of 8-12 weeks. In some embodiments, the first treatment period has a duration of 8 - 12 weeks or 1 - 8 weeks or 2 - 12 weeks. In some instances, the first treatment period is at least one week. In some instances, the pegylated interferon lambda is administered once a week. In some instances, the pegylated interferon lambda is administered twice per week.

In some embodiments, the pegylated interferon lambda-1a is administered at a dose of 180 micrograms once a week (QW). In some embodiments, the pegylated interferon lambda-1a is administered at a dose of 120 micrograms QW. In some embodiments (i) 160 - 180 micrograms pegylated interferon lambda-1a is administered per week for a first treatment period and then 150 - 170 micrograms per week for a second treatment period; or (ii) 180 micrograms per week for a first treatment period and then between 120 - 170 micrograms per week for a second treatment period, wherein the doses for each of (i) and (ii) may be divided into more than one dose per week.

In some embodiments, the method comprises administering the pegylated interferon lambda-1a at a dose of 180 micrograms QW for a first treatment period and then at a dose of 120 micrograms QW for a second treatment period. In some embodiments, the method comprises administering the pegylated interferon lambda-1a at a dose of 120 micrograms QW for a first treatment period and then at a dose of 80 micrograms QW for a second treatment period. In some embodiments, the method further comprises administering the pegylated interferon lambda-1a at a dose of 80 micrograms QW for a third treatment period. In some embodiments, the method comprises administering the pegylated interferon lambda-1a at a dose of 180 micrograms QW for a first treatment period and then at a dose of 120 micrograms QW for a second treatment period followed by administering a dose of 60 - 110 micrograms QW for a third treatment period.

In some embodiments, the method comprises administering the pegylated interferon lambda-1a at a first dose of 180 micrograms QW for a first treatment period, at a second dose of 120 micrograms QW for a second treatment period, and at a third dose of 80 - 110 micrograms QW for a third treatment period. In some embodiments, the first treatment period has a duration of at least 8 weeks. In some embodiments, the first treatment period has a duration of 8 - 12 weeks or 1 - 8 weeks or 2 - 12 weeks.

In some embodiments, the symptoms of coronavirus infection include one or more of: pneumonia (e.g., lungs inflamed and the tiny sacs where oxygen moves from the air to the blood were filling with water), fever, cough, shortness of breath, and muscle ache. Other symptoms may include confusion, headache, and sore throat.

In some embodiments, treatment results in a reduction of coronavirus viral load in the subject of at least 2.0 log10 coronavirus RNA copies/mL serum. In some embodiments, treatment results in a coronavirus viral load that is below the level of detection. In some embodiments, prior to the onset of treatment, the subject has a serum alanine aminotransferase (ALT) level that is above the upper limit of normal (ULN), and the course of treatment results in an improvement in serum ALT level in the subject to a level that is below the ULN.

In some embodiments, prior to treatment, the subject has a baseline viral load of up to about 10⁴ coronavirus RNA copies per mL sample.

In some embodiments, subjects having a low viral load have a higher percentage of BLQ response at 48 weeks and at 24 weeks post treatment.

In one embodiment, the interferon lambda 180 µg treatment group, response rates differed between subjects with high (> 6 logs) versus low (≤ 6 logs) baseline viral load. In one embodiment, at week 48, 38 - 43% and 33 - 40% of subjects with high versus low baseline viral loads respectively, reached coronavirus RNA levels BLQ.

Other aspects and embodiments are described throughout this disclosure.

### BRIEF DESCRIPTION OF THE DRAWINGS

FIG. 1. shows evaluation of treatment of human primary airway epithelial cells infected with SARS-CoV-2 with pegylated interferon lambda according to various aspects of this disclosure.

FIGS. 2A-2C shows evaluation of prevention and intervention strategies against SARS-CoV-2 MA infection in mice according to various aspects of this disclosure. FIG. 2A. shows human primary airway epithelial cells pretreated for 24 hrs with peg-IFN-λ1 followed by infection with SARS-CoV-2 WT according to aspects of this disclosure. Infectious virus in apical washes from 48 hours post infection was titered. Remdesivir (RDV) was used as positive control. Dotted line represents limit of detection. Undetected samples are plotted at half the limit of detection. This study was repeated in cells from two unique human donors. FIG. 2B and FIG. 2C shows results of 12-week-old female BALB/c mice were subcutaneously treated with vehicle (gray) or with 2 µg peg-IFN-λ1 prophylactically (orange) or therapeutically (purple) and infected with SARS-CoV-2 MA according to aspects of this disclosure. FIG. 2B shows lung viral titer; dotted line represents limit of detection. FIG. 2C shows nasal turbinate viral titer; dotted line represents limit of detection. The line represents the mean and error bars represent standard error of the mean. Asterisk denotes p<0.05.

FIGS. 3A - 3H show decline in viral load (measured as SARS-CoV-2 RNA copies/mL) over time among participants in the trial of Example 6 according to various aspects of this disclosure. FIG. 3A shows viral load in the days following injection. The mean SARS-CoV-2 RNA viral load was lower in the pegylated interferon lambda treatment group than in the placebo group at Day 7 (p=0.081) and at Day 0 (p=0.11). FIG. 3B shows the log reduction in viral load in the days following injection. The mean log decline in RNA viral load was significantly greater in patients treated with pegylated interferon lambda than in those treated with placebo from Day 5 onwards. The statistical significance of the difference in mean viral load decline for days is as follows: for Day 3, p=0.14; for Day 5, p=0.013; for Day 7, p=0.004; and for Day 14, p=0.048. FIG. 3C shows viral load in the days following injection in the pegylated interferon treatment (n=19) group and the placebo group (n=16), stratified by subjects having a baseline viral load above 10⁶ SARS-CoV-2 RNA copies/mL. The mean SARS-CoV-2 RNA viral load was significantly lower in the pegylated interferon lambda treatment group than in the placebo group at Day 7 (p=0.017). FIG. 3D shows the mean log decline in SARS-CoV-2 RNA viral load in the days following injection in the pegylated interferon treatment (n=19) group and the placebo group (n=16), stratified by subjects having a baseline viral load above 10⁶ SARS-CoV-2 RNA copies/mL. The statistical significance of the difference in mean viral load decline for days is as follows: for Day 3, p=0.042; for Day 5, p=0.029; for Day 7, p=0.004; and for Day 14, p=0.039. FIG. 3E shows viral load in the days following injection in the pegylated interferon treatment (n=11) group and the placebo group (n=14), stratified by subjects having a baseline viral load below 10⁶ SARS-CoV-2 RNA copies/mL. The difference in mean SARS-CoV-2 RNA viral load at Day 7 was relatively small (p=0.79). FIG. 3F shows the mean log decline in SARS-CoV-2 RNA viral load in the days following injection in the pegylated interferon treatment (n=11) group and the placebo group (n=14), stratified by subjects having a baseline viral load below 10⁶ SARS-CoV-2 RNA copies/mL. The mean log decline in SARS-CoV-2 RNA viral load was significantly lower in the pegylated interferon lambda treatment group than in the placebo group at Day 7 (p=0.20). FIG. 3G shows viral load in the days following injection in the pegylated interferon treatment group and the placebo group, stratified by subjects having a detectable baseline viral load. FIG. 3H shows the mean log decline in SARS-CoV-2 RNA viral load in the days following injection in the pegylated interferon treatment group and the placebo group, stratified by subjects having a detectable baseline viral load.

FIG. 4 shows the odds of clearance by Day 7 according to baseline viral load in the pegylated interferon lambda group compared to the placebo group for every baseline viral load in log IU/mL according to aspects of this disclosure.

FIGS. 5A - 5C show the proportion of patients negative for SARS-CoV-2 RNA in the days following injection among participants in the trial of Example 6 according to various aspects of this disclosure. FIG. 5A shows the proportion of patients negative for SARS-CoV-2 RNA per day post-injection across all patients. In the graph, for each day, the interferon lambda group is shown on the left, and the placebo group is shown on the right. The proportion of patients who tested negative was significantly greater in the pegylated interferon lambda than the placebo group at Day 7 (p=0.15). FIG. 5B shows the proportion of patients negative for SARS-CoV-2 RNA per day post-injection for patients having a baseline viral load above 10⁶ SARS-CoV-2 RNA copies/mL. In the graph, for each day, the interferon lambda group is shown on the left, and the placebo group is shown on the right. The proportion of patients who tested negative was significantly greater in the pegylated interferon lambda than the placebo group at Day 7 (p=0.013). FIG. 5C shows the proportion of patients negative for SARS-CoV-2 RNA per day post-injection for patients having a baseline viral load below 10⁶ SARS-CoV-2 RNA copies/mL. For each day, the interferon lambda group is shown on the left, and the placebo group is shown on the right. The proportion of patients who tested negative was significantly greater in the pegylated interferon lambda than the placebo group at Day 7 (p=0.40). FIG. 5D shows the proportion of patients with positive anti-SARS-CoV-2 S protein IgG antibodies at Days 0, 3, 7 and 14 post-injection, stratified by baseline viral load above or below 10⁶ copies/mL and treatment group. In the graph, for each day, the interferon lambda group is shown on the left, and the placebo group is shown on the right.

FIG. 6 shows time to clearance by group among participants in the trial of Example 6 with baseline viral load above 10⁶ SARS-CoV-2 RNA copies/mL, comparing the pegylated interferon lambda group and the placebo group according to various aspects of this disclosure. The curves are compared using the log-rank test, and the median time to clearance with 95%CI is shown for each group.

FIG. 7A shows symptom severity by symptom category and treatment group over time according to various aspects of this disclosure. The proportion of participants reporting no, mild, moderate or severe symptoms is shown for the pegylated interferon lambda and the placebo group. In the graph, for each day, the severity grades are, from top to bottom in each bar, none, mild, moderate, and severe. Among both the interferon lambda group and the placebo group, no severe symptoms were reported on Day 7. Among the placebo group, no moderate or severe symptoms were reported on Days 10 and 14. Symptoms were grouped into categories and the most severe ranking of any symptom in the category was used for each participant at each day. Symptoms declined in both groups over time (p<0.0001) and there was no difference in overall symptoms (p=0.11) or the decline in symptoms between groups (p=0.32).

FIG. 7B shows the proportion of participants in the trial of Example 6 with fever above 38° C., stratified by day and group, according to various aspects of this disclosure. In the graph, for each day, the temperatures are, from top to bottom in each bar, <38, 38-39, and 39-40. Temperatures of 38-39 were observed on Days 0, 0.5, 4, and 6 in the interferon lambda group and on all days in the placebo group. Temperatures of 39-40 were observed on Days 0, 2, 3, 4, 5, 6, 7, 10, 12, and 14 in the interferon lambda group and on no days in the placebo group.

FIGS. 8A - 8C shows laboratory values over time by treatment group according to various aspects of this disclosure. For each value illustrated in FIGS. 8A - 8C, the normal laboratory range is shown by a dashed line, the column on the left for each time point indicates patients in the interferon lambda arm, and the column on the right for each time point indicates patients in the placebo arm. The median (IQR) for hematological, hepatic and inflammatory markers are shown at Day 0, 3, 7 and 14. FIG. 8A shows laboratory values for hematological markers. In each of the graphs, for each day, the interferon lambda group data is shown on the left and the placebo group data is shown on the right. In FIG. 8A, WBC refers to white bloody cells; Neutrophils refers to absolute neutrophil count; and Lymphocytes refers to absolute lymphocyte count. FIG. 8B shows laboratory values for hepatic markers. In FIG. 8b, ALT refers to alanine aminotransferase; AST refers to aspartate aminotransferase; and ALP refers to alkaline phosphatase. FIG. 8C shows laboratory values for inflammatory markers. In FIG. 8C, CRP refers to c-reactive protein; and LDH refers to lactate dehydrogenase.

FIG. 9 shows a Schedule of Events for the trial described in Example 7 according to various aspects of this disclosure. Urine pregnancy tests (marked *) are administered for women of child-bearing age. The safety lab tests (marked **) include CBC, AST, ALT, ALP, creatinine, electrolyte, amylase/lipase, bilirubin, albumin, random glucose testing. Cytokine and inflammatory markers (marked ***) include fortin, lactate dehydrogenase, D Dimer, C reactive protein, creatine kinase. The research sample for plasma (marked ****) is collected and stored for future use. Collection of a sample for genetic testing (IFNL4), a peripheral blood mononuclear cell (PBMC) sample, and a dried blood sample (each marked *****) are each optional for those who agreed.

### DETAILED DESCRIPTION

### I. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

All numerical designations, e.g., pH, temperature, time, concentration, and molecular weight, including ranges, are approximations which are varied (+) or (-) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “administration” refers to introducing a compound, a composition, or an agent of the present disclosure into a host, such as a human. In the context of the present disclosure, one preferred route of administration of the agents is subcutaneous administration. Other routes of administration include intravenous administration and oral administration.

The term “baseline,” unless otherwise specified or apparent from context, refers to a measurement (of, e.g., viral load, subject condition, ALT level) made prior to a course of therapy.

The term “comprising” is intended to mean that the compounds, compositions and methods include the recited elements, but does not exclude others. “Consisting essentially of” when used to define compounds, compositions and methods, shall mean excluding other elements that would materially affect the basic and novel characteristics of the claimed invention. Embodiments defined by each of these transition terms are within the scope of this invention.

The terms “course of treatment” and “course of therapy” are used interchangeably and refer to the medical interventions made after a subject is diagnosed, e.g., as being infected with coronavirus and in need of medical intervention. Medical interventions include, without limitation, the administration of drugs for a period of time, typically, for coronavirus infected subjects, at least one and typically several or many months or even years.

In the context of this disclosure, the terms “Coronavirus infection” and “COVID-19 infection” with respect to a human (host) refers to the fact that the host is suffering from Coronavirus infection and from an infection of SARS-CoV-2, respectively. Typically, an coronavirus infected human host will have a viral load of coronavirus of about 2 log10 copies per milliliter in the severe group and 10 log10 copies per milliliter; from about 1 log10 copies per milliliter in the severe group and 15 log10 copies per milliliter; 3 log10 copies per milliliter in the severe group and 5 log10 copies per milliliter; 4 log10 copies per milliliter in the severe group and 7.5 log10 copies per milliliter; 2 log10 copies per milliliter in the severe group and 8 log10 copies per milliliter. The sample may be from throat swabs, nasopharyngeal-swab, sputum or tracheal aspirate, urine fecal, and blood samples.

Known coronavirus isolates include SARS-CoV-2 (also referred to as “coronavirus 2019-nCoV” and “2019-nCoV”, new coronavirus identified in 2019 causing COVID-19) and variants thereof (e.g., the 501.V2 variant, the B.1.1.248 variant, the Cluster 5 variant, and the B.1.1.7 201/501Y.V1 variant), Canine coronavirus, Canine enteric coronavirus (strain INSAVC-1), Canine enteric coronavirus (strain K378), Feline coronavirus, Feline enteric coronavirus (strain 79-1683), Feline infectious peritonitis virus (FIPV), Human coronavirus 229E, Porcine epidemic diarrhea virus, Porcine epidemic diarrhea virus (strain Br1/87), Porcine epidemic diarrhea virus (strain CV777), Transmissible gastroenteritis virus, Porcine respiratory coronavirus, Porcine transmissible gastroenteritis coronavirus (STRAIN FS772/70), Porcine transmissible gastroenteritis coronavirus (strain Miller), Porcine transmissible gastroenteritis coronavirus (strain Neb72-RT), Porcine transmissible gastroenteritis coronavirus (STRAIN PURDUE), Bovine coronavirus, Bovine coronavirus (STRAIN F15), Bovine coronavirus (strain G95), Bovine coronavirus (STRAIN L9), Bovine coronavirus (strain LSU-94LSS-051), Bovine coronavirus (STRAIN LY-138), Bovine coronavirus (STRAIN MEBUS), Bovine coronavirus (strain OK-0514-3), Bovine coronavirus (strain Ontario), Bovine coronavirus (STRAIN QUEBEC), Bovine coronavirus (STRAIN VACCINE), Bovine enteric coronavirus (strain 98TXSF-110-ENT), Canine respiratory coronavirus, Chicken enteric coronavirus, Human coronavirus OC43, Murine hepatitis virus, Murine coronavirus (strain DVIM), Murine hepatitis virus (strain A59), Murine hepatitis virus (strain JHM), Murine hepatitis virus (strain S), Murine hepatitis virus strain 1, Murine hepatitis virus strain 2, Murine hepatitis virus strain 3,Murine hepatitis virus strain 4, Murine hepatitis virus strain ML-11, Porcine hemagglutinating encephalomyelitis virus, Porcine hemagglutinating encephalomyelitis virus (strain 67N), Porcine hemagglutinating encephalomyelitis virus (strain IAF- 404), Puffinosis virus Rat coronavirus, Rat coronavirus (strain 681), Rat coronavirus (strain NJ), Rat sialodacryoadenitis coronavirus, Turkey coronavirus Turkey coronavirus (strain Indiana), Turkey coronavirus (strain Minnesota), Turkey coronavirus (strain NC95), Avian infectious bronchitis virus, Avian infectious bronchitis virus (STRAIN 6/82), Avian infectious bronchitis virus (strain Arkansas 99), Avian infectious bronchitis virus (strain Beaudette CK), Avian infectious bronchitis virus (strain Beaudette M42), Avian infectious bronchitis virus (strain Beaudette US), Avian infectious bronchitis virus (strain Beaudette), Avian infectious bronchitis virus (strain D1466), Avian infectious bronchitis virus (strain D274), Avian infectious bronchitis virus (strain D3896), Avian infectious bronchitis virus (strain D41), Avian infectious bronchitis virus (strain DE072), Avian infectious bronchitis virus (strain GRAY), Avian infectious bronchitis virus (strain H120), Avian infectious bronchitis virus (strain H52), Avian infectious bronchitis virus (strain KB8523), Avian infectious bronchitis virus (strain M41), Avian infectious bronchitis virus (strain PORTUGAL/322/82), Avian infectious bronchitis virus (strain SAlB20), Avian infectious bronchitis virus (strain UK/123/82), Avian infectious bronchitis virus (strain UK/142/86), Avian infectious bronchitis virus (strain UK/167/84), Avian infectious bronchitis virus (strain UK/183/66), Avian infectious bronchitis virus (strain UK/68/84), Avian infectious bronchitis virus (strain V18/91), Avian infectious bronchitis virus (strain Vic S), Avian infectious laryngotracheitis virus, SARS coronavirus, SARS coronavirus, Beijing ZY-2003, SARS coronavirus BJ01, SARS coronavirus BJ02, SARS coronavirus BJ03, SARS coronavirus BJ04, SARS coronavirus CUHK-Su10, SARS coronavirus CUHK-W1, SARS coronavirus Frankfurt 1, SARS coronavirus GZ01, SARS coronavirus HKU-39849, SARS coronavirus Hong Kong ZY-2003, SARS coronavirus Hong Kong/03/2003, SARS coronavirus HSR 1, SARS coronavirus Sin2500, SARS coronavirus Sin2677, SARS coronavirus Sin2679, SARS coronavirus Sin2748, SARS coronavirus Sin2774, SARS coronavirus Taiwan, SARS coronavirus Taiwan JC-2003, SARS coronavirus Taiwan TC1, SARS coronavirus Taiwan TC2, SARS coronavirus Tor2, SARS coronavirus TW1, SARS coronavirus TWC, SARS coronavirus Urbani, SARS coronavirus Vietnam, SARS coronavirus ZJ-HZ01, SARS coronavirus ZJ01, unclassified coronaviruses, Bovine respiratory coronavirus (strain 98TXSF-110-LUN), Human enteric coronavirus 4408, Enteric coronavirus, Equine coronavirus, and Equine coronavirus NC99.

The term “Lower Limit of Quantification” refers to the lowest concentration of a substance of analyte (e.g., a viral titer) that can be reliably quantified by a particular assay within a stated confidence limit.

The terms “subject,” “host,” or “subject,” are used interchangeably and refer to a human infected with coronavirus, including subjects previously infected with coronavirus in whom virus has cleared.

The term “pharmaceutical composition” is meant to encompass a composition suitable for administration to a subject. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or subjects in need thereof via a number of different routes of administration including oral, intravenous, buccal, rectal, parenteral, intraperitoneal, intradermal, intratracheal, intramuscular, subcutaneous, inhalational, and the like.

A “sustained reduction” of coronavirus viral load means a reduction of viral load (e.g., a decrease of at least 0.5 log₁₀ copies/ml of coronavirus in a sample, a decrease of at least 1 log₁₀ copies/ml of coronavirus in a sample, a decrease of at least 1.5 log₁₀ copies/ml of coronavirus in a sample, at least 2.0 log₁₀ copies/ml of coronavirus in a sample or at least 2.5 log₁₀ copies/ml of coronavirus in a sample, or a decrease in coronavirus to undetectable levels) for a period time (e.g., 1 month, 3 months, 6 months, 1 year, longer, forever or until a subsequent coronavirus infection). The sustained reduction may be a period of time during which the course of treatment is still ongoing or a period of time after the course of treatment is finished.

The term “therapeutically effective amount” as used herein refers to that amount of an embodiment of the agent (e.g., a compound, inhibitory agent, or drug) being administered that will treat to some extent a disease, disorder, or condition, e.g., relieve one or more of the symptoms of the disease, i.e., infection, being treated, and/or that amount that will prevent, to some extent, one or more of the symptoms of the disease, i.e., infection, that the subject being treated has or is at risk of developing.

The terms “treatment,” “treating,” and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a human subject, and includes: (a) reducing the risk of occurrence of the disease in a subject determined to be predisposed to the disease but not yet diagnosed as infected with the disease, (b) impeding the development of the disease, and/or (c) relieving the disease, e.g., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of an agent that provides for enhanced or desirable effects in the subject (e.g., reduction of viral load, reduction of disease symptoms, etc.).

The terms “undetectable” or “below the level of detection” or “BLD”, as used with reference to coronavirus RNA levels, means that no coronavirus RNA copies can be detected by the assay methodology employed. In some embodiments, the assay is quantitative RT-PCR.

The term “durable virologic response” or “DVR” as used herein refers to post-treatment response in a subject of coronavirus RNA below the limit of quantitation (BLQ) within one or more weeks after the end of treatment, or from between 2-12 weeks of ending treatment from between 12 and 24 weeks after ending treatment, from 1 day to 2-weeks; or from 12 - 48 weeks after ending treatment.

### II. Methods of Treatment

In one aspect, the present disclosure provides methods of treating Coronavirus infection by administering interferon lambda therapy to a coronavirus-infected subject. In some embodiments, a pegylated form of interferon lambda (e.g., pegylated interferon lambda-1a) is administered. In some embodiments, subjects receiving interferon lambda therapy (e.g., pegylated interferon lambda therapy) are also treated with an antiviral nucleoside or nucleotide analog (e.g., an anti-HBV nucleotide or nucleoside analog). In some embodiments, subjects receiving interferon lambda therapy (e.g., pegylated interferon lambda therapy) are not administered an antiviral nucleoside or nucleotide analog therapy.

### Interferon Lambda

Interferons are polypeptides that inhibit viral replication and cellular proliferation and modulate immune response. Interferons are produced as part of the innate immune response to viral infections, driving the induction of a broad array of host of genes with antiviral, antiproliferative and immuno-regulatory properties. Based on the type of receptor through which they signal, human interferons have been classified into three major types (Types I, II, and III). Both Type I and Type III IFNs signal through the JAK-STAT pathway to drive ISG induction with comparable antiviral activity, however their systemic effects differ markedly due to the use of distinct receptors with different tissue distributions. All type I IFNs bind to a specific cell surface receptor complex known as the IFN-alpha receptor (IFNAR) that consists of IFNAR1 and IFNAR2 chains. The type I interferons present in humans are IFN-alpha, IFN-beta, IFN-epsilon, and IFN-omega. The type I IFN receptor is highly expressed on all cells in the body. Type II IFNs bind to IFN-gamma receptor (IFNGR) that consists of IFNGR1 and IFNGR2 chains. The type II interferon in humans is IFN-gamma. The type III interferon group includes three IFN-lambda molecules called IFN-lambda1, IFN-lambda2, and IFN-lambda3 (also called IL29, IL28A, and IL28B, respectively). These IFNs signal through a receptor complex consisting of IL10R2 (also called CRF2-4) and IFNLR1 (also called CRF2-12). Type III IFNs exert a similar antiviral state to IFN-alpha and IFN-beta but use a distinct receptor complex with high expression levels limited to epithelial cells in the lung, liver, and intestine as well as very limited expression in hematopoietic and central nervous system cells. See Syedbasha M & Egli A, “Interferon Lambda: Modulating Immunity in Infectious Diseases,” Front. Immunol. 2017; 8:119, doi:10.3389/fimmu.2017.00119. The more limited receptor expression profile can result in fewer systemic side effects. For example, interferon-lambda has been found to control respiratory viral infections in mice without the risk of promoting cytokine storm syndrome, as has been seen with Type I interferon treatment.

The term “interferon-lambda” or “IFN-λ” as used herein includes naturally occurring IFN-λ; synthetic IFN-λ; derivatized IFN-λ (e.g., PEGylated IFN-λ, glycosylated IFN-λ, and the like); and analogs of naturally occurring or synthetic IFN-λ. In some embodiments, an IFN-λ is a derivative of IFN-λ that is derivatized (e.g., chemically modified relative to the naturally occurring peptide) to alter certain properties such as serum half-life. As such, the term “IFN-λ” includes IFN-λ derivatized with polyethylene glycol (“PEGylated IFN-λ”), and the like. PEGylated IFN-λ (e.g., PEGylated IFN-λ-1a), and methods for making same, is discussed in, e.g., U.S. Pat. Nos. 6,927,040, 7,038,032, 7,135,170, 7,157,559, and 8,980,245; and PCT Publication Nos. WO 2005/097165, WO 2007/012033, WO 2007/013944 and WO 2007/041713; all of which are herein incorporated by reference in their entirety. In some embodiments, the IFN-λ is an IFN-λ as disclosed in PCT/US2017/018466, which is incorporated by reference herein in its entirety. In some embodiments, the pegylated IFN-λ-1a has the structure described in US 7,157,559, which is incorporated by reference herein in its entirety. IFN-λ has been found to be effective for acute respiratory disease due to the high expression of the IFN-λ receptor in lung epithelia.

As described in this disclosure, IFN-λ is effective as a therapeutic treatment of a SARS-CoV-2 infection including in patients with COVID-19. Without being bound by theory, it is believed that the effectiveness of IFN-λ is due to the high expression of the IFN-λ receptor in the lungs, intestine, and liver. This is consistent with the intestinal and hepatic involvement documented in patients with COVID-19. In some embodiments, this therapeutic treatment provides the benefit of reduced incidence of or the symptoms (intensity or kind) of cytokine storm syndrome in patients with COVID-19. This is consistent with the lack of the lambda receptor on hematopoietic cells. See Zhang W et al., “Molecular and serological investigation of 2019-nCoV infected patients: implication of multiple shedding routes,” Emerg. Microbes Infect. 2020; 9(1):386-389. doi:10.1080/22221751.2020.1729071.

In some embodiments, an interferon for use in a therapeutic method as described herein is a pegylated IFN-λ1 (e.g., pegylated IFN-λ-1a), pegylated IFN-λ-2, or pegylated IFN-λ-3. In some embodiments, the interferon is pegylated IFN-λ1 (e.g., pegylated IFN-λ-1a).

In some embodiments, pegylated IFN-λ1 has the amino acid sequence shown below (lines show intrachain disulfide bonds) [SEQ ID NO:1]:

[symbol]

### Subject Population

In some embodiments, a subject to be treated with interferon lambda therapy as described herein is a subject having a Coronavirus infection, an acute Coronavirus infection, or a long term (persistent) Coronavirus infection. In some cases, the subject to be treated is identified as having a Coronavirus infection by a positive coronavirus antibody (Ab) test and/or detectable coronavirus RNA by qRT-PCR. In some instances, the molecular or antibody-based testing is performed using point-of-care (POC) testing, such as, e.g., Abbott ID NOW™ and/or Assure® COVID-19 IgG/IgM Rapid Test Device. In some embodiments, the subject to be treated has a Coronavirus infection of at least 1 month documented by a positive coronavirus Ab test, and/or detectable coronavirus RNA by qRT-PCR. In some embodiments, a subject to be treated with a therapeutic method described herein is a subject having an acute Coronavirus infection, one that is newly diagnosed or otherwise believed not to have existed in the subject for more than one week. Diagnosis of infection with SARS-CoV-2 and/or COVID-19 is described herein.

In some embodiments, a subject to be treated has a positive test for coronavirus infection. In some embodiments, the Coronavirus infection is an infection of a subject with SARS-CoV-2 or a variant thereof. In some embodiments, the viral load is detectable. In some embodiments, the viral load is at least 10² coronavirus RNA copies per mL of sample (e.g., throat swabs, nasopharyngeal-swab, sputum or tracheal aspirate, urine fecal, and blood samples). In some embodiments, the viral load is at least 10² IU/mL of sample, e.g., at least 10³ coronavirus RNA copies per mL or at least 10³ IU/mL sample, at least 10⁴ coronavirus RNA copies per mL or at least 10⁴ IU/mL sample, at least 10⁵ coronavirus RNA copies per mL or at least 10⁵ IU/mL sample, at least 10⁶ coronavirus RNA copies per mL or at least 10⁶ IU/mL sample, at least 10⁷ coronavirus RNA copies per mL or at least 10⁷ IU/mL sample, or at least 10⁸ coronavirus RNA copies per mL or at least 10⁸ IU/mL sample. In some embodiments, coronavirus viral load is measured using serum samples from the subject. In some embodiments, coronavirus viral load is measured using plasma samples from the subject. In some embodiments, viral load is measured by quantitative RT-PCR. qRT-PCR assays for quantification of coronavirus RNA in sample are known in the art, e.g., as described above. In some embodiments, a subject to be treated has a baseline viral load that is up to about 10⁴ coronavirus RNA copies per mL sample or up to about 10⁴ IU/mL sample. In some embodiments, a subject to be treated has a baseline viral load that is up to about 10⁵ coronavirus RNA copies per mL sample or up to about 10⁵ IU/mL sample. In some embodiments, a subject to be treated has a baseline viral load that is up to about 10⁶ coronavirus RNA copies per mL sample or up to about 10⁶ IU/mL sample.

In some embodiments, coronavirus viral load is measured using samples from the subject. In some embodiments, coronavirus viral load is measured using a serum or plasma sample from the subject. In some embodiments, viral load is measured by quantitative RT-PCR. qRT-PCR assays for quantification of coronavirus RNA in samples are known in the art, e.g., as described herein. In some instances, the sample from the subject is a respiratory sample, including but not limited to a nasopharyngeal aspirate or wash, an oropharyngeal aspirate or wash, a nasopharyngeal swab, an oropharyngeal swab, a broncheoalveolar lavage, a tracheal aspirate, and/or sputum.

In some embodiments, a subject to be treated exhibits one or more symptoms of coronavirus infection, e.g., fever, cough, shortness of breath. In some instances, the subject exhibits one or more of leukopenia, leukocytosis, lymphopenia, elevated alanine aminotransferase, and/or elevated aspartate aminotransferase levels.

In some embodiments, the subject exhibits a DNA sequence variation, such as a a single nucleotide polymorphism. In some cases, for example, the subject may exhibit a single nucleotide polymorphism near the interleukin 28B (IL28B) gene. In some instances, this single nucleotide polymorphism is strongly associated with response to treatment. In some cases, the single nucleotide polymorphism corresponds to an mRNA transcript that codes for interferon lambda 4 (IFNL4). See Prokunina-Olsson L et al., “A variant upstream of IFNL3 (IL28B) creating a new interferon gene IFNL4 is associated with impaired clearance of hepatitis C virus,” Nat. Genet., February 2013; 45(2):164-71. doi:10.1038/ng.2521.

In some embodiments, the subject to be treated will not have had any of the following: treatment with interferons (IFNs) immunomodulators and/or immunosuppressive or B-cell depleting medications within 12 months before screening; previous use of Interferon Lambda; history or evidence of any intolerance or hypersensitivity to IFNs; respiratory infection requiring invasive or non-invasive ventilatory support (bipap or intubation and mechanical ventilation); participation in a clinical trial with use of any investigational drug within 30 days before screening; or history of any of the following diseases or conditions: advanced or decompensated liver disease (presence or history of bleeding varices, ascites, encephalopathy or hepato-renal syndrome); immunologically mediated disease (e.g., rheumatoid arthritis, inflammatory bowel disease, severe psoriasis, systemic lupus erythematosus) that requires more than intermittent nonsteroidal anti-inflammatory medications for management or that requires use of systemic corticosteroids in the 6 months before screening (inhaled asthma medications are allowed); retinal disorder or clinically relevant ophthalmic disorder; or any malignancy within 5 years before screening.

In some embodiment, the subject to be treated may have had one or more of the following: a superficial dermatologic malignancy (e.g., squamous cell or basal cell skin cancer treated with curative intent); cardiomyopathy, significant ischemic cardiac or cerebrovascular disease (including history of angina, myocardial infarction, or interventional procedure for coronary artery disease), or cardiac rhythm disorder; chronic pulmonary disease (e.g., chronic obstructive pulmonary disease) associated with functional impairment; pancreatitis; severe or uncontrolled psychiatric disorder; active seizure disorder defined by either an untreated seizure disorder or continued seizure activity within the preceding year despite treatment with anti-seizure medication; bone marrow or solid organ transplantation; or any of the following abnormal laboratory test in the 12 months prior to enrollment: platelet count <90,000 cells/mm³; white blood cell (WBC) count <3,000 cells/mm³; absolute neutrophil count (ANC) <1,500 cells/mm³; hemoglobin <11 g/dL for women and <12 g/dL for men; estimated creatinine clearance (CrCl) < 50 mL/min by Cockroft-Gault formulation; ALT and/or ALT levels > 10 times the upper limit of normal; bilirubin level ≥ 2.5 mg/dL unless due to Gilbert’s syndrome; serum albumin level <3.5 g/dL; or an international normalized ratio (INR) ≥1.5 (except patients maintained on anticoagulant medications)

### Interferon Lambda Dosing Regimens

In some embodiments, interferon lambda therapy comprises administering to the subject interferon lambda (e.g., pegylated interferon lambda-1a) at a dose of 180 micrograms (mcg) per week, 120 mcg per week, 110 mcg per week, 100 mcg per week, 90 mcg per week, 80 mcg per week, 120 - 70 mcg per week, 200 - 120 mcg per week, or 170 - 130 mcg per week. In some embodiments, interferon lambda is administered at a dose of 180 mcg QW. In some embodiments, interferon lambda is administered at a dose of 90 mcg two times per week. In some embodiments, interferon lambda is administered at a dose of 90 mcg every 3 - 4 days. In some embodiments, interferon lambda is administered at a dose of 80 mcg two times per week. In some embodiments, interferon lambda is administered at a dose of 80 mcg every 3 - 4 days. In some embodiments, interferon lambda is administered at a dose of 100 - 70 mcg two time per week. In some embodiments, interferon lambda is administered at a dose of 100 - 70 mcg every 3 - 4 days. In some embodiments, interferon lambda is administered at a dose of 120 mcg QW. In some embodiments, interferon lambda is administered at a dose of 80 mcg QW.

In some embodiments, a subject being treated for Coronavirus infection receives an adjustment in the dosing regimen of the interferon lambda therapy during the course of treatment. In some embodiments, the subject receives a dose reduction of interferon lambda, in that one or more later doses is a lower dose than one or more earlier doses. In some embodiments, a dose is reduced if the subject exhibits unacceptable side effects. In some embodiments, a subject may receive multiple dose reductions during the course of treatment with interferon lambda.

In some embodiments, the dosage administered to the subject is not reduced before 2 weeks of treatment at the first dosage (e.g., at a first dosage of 180 mcg QW), or before 3 week, or 2 weeks, or 3 weeks, or 4 weeks, or 5 weeks, or 6 weeks, or 7 weeks of treatment at the first dosage. In some embodiments, the dosage administered to the subject is not reduced before 9-12 weeks of treatment at the first dosage (e.g., at a first dosage of 180 mcg QW).

The interferon lambda therapy may comprise administering to the subject interferon lambda at differing doses between two or more treatment periods. In some embodiments, the interferon lambda therapy comprises administering to the subject interferon lambda at a dose of 180 micrograms per week for a first treatment period followed by administering to the subject interferon lambda at a dose of 120 micrograms per week for a second treatment period. In some embodiments, the length of time for the first treatment period is the same as the length of time for the second treatment period. In some embodiments, the first treatment period and the second treatment period are different lengths of time. In some embodiments, the first treatment period (i.e., interferon lambda at a dose of 180 mcg per week) is longer than the second treatment period (i.e., interferon lambda at a dose of 120 mcg per week). In some embodiments, the second treatment period (i.e., interferon lambda at a dose of 120 mcg per week) is longer than the first treatment period (i.e., interferon lambda at a dose of 180 mcg per week). In some embodiments, the interferon lambda therapy further comprises administering to the subject interferon lambda at a dose of 110 - 80 micrograms per week for a third treatment period. In some embodiments, the length of time for the third treatment period is the same as the length of time for the first and/or second treatment period. In some embodiments, the third treatment period and the first and/or second treatment period are different lengths of time. In some embodiments, the third treatment period (i.e., interferon lambda at a dose of 110 - 80 mcg per week) is longer than the first and/or second treatment period. In some embodiments, the third treatment period (i.e., interferon lambda at a dose of 80 mcg per week) is shorter than the first and/or second treatment period.

In some embodiments, the interferon lambda therapy comprises administering interferon lambda at a dose of 120 micrograms per week for a first treatment period followed by administering interferon lambda at a dose of 110 - 80 micrograms per week for a second treatment period. In some embodiments, the length of time for the first treatment period is the same as the length of time for the second treatment period. In some embodiments, the first treatment period and the second treatment period are different lengths of time. In some embodiments, the first treatment period (i.e., interferon lambda at a dose of 120 mcg per week) is longer than the second treatment period (i.e., interferon lambda at a dose of 80 mcg per week). In some embodiments, the second treatment period (i.e., interferon lambda at a dose of 80 mcg per week) is longer than the first treatment period (i.e., interferon lambda at a dose of 120 mcg per week).

In some embodiments, the interferon lambda therapy comprises administering interferon lambda at a first dose of 180 micrograms QW for a first treatment period, at a second dose of 170 - 120 micrograms QW for a second treatment period, and at a third dose of 110 - 80 micrograms QW for a third treatment period. In some embodiments, the first treatment period has a duration of at least 8 weeks, or from 1 - 8 weeks, or from 1 - 12 weeks. In some embodiments, the first treatment period has a duration of 8 - 12 weeks.

In some embodiments, the interferon lambda therapy comprises administering interferon lambda at a first dose of 160 - 180 micrograms per week for a first treatment period, at a second dose of 170 - 120 micrograms per week for a second treatment period, and at a third dose of 110 - 60 micrograms per week for a third treatment period. In some embodiments, the first treatment period has a duration of at least 8 weeks, or from 1 - 8 weeks, or from 1 - 12 weeks. In some embodiments, the first treatment period has a duration of 8 - 12 weeks. Doses may be given in multiple dose per week with the number of micrograms equaling the weekly dose.

In some embodiments, a treatment period (e.g., a first treatment period, second treatment period, and/or third treatment period) is at least 1 week in duration, e.g., at least 2, 3, 4 weeks or longer. In some embodiments, a treatment period (e.g., a first treatment period, second treatment period, and/or third treatment period) is at least 2 weeks in duration, e.g., at least 4, 6, 8, 10, 12, 16, 20, 24, 28, 32, 36, 40, 44, 48 weeks, or longer. In some embodiments, a treatment period is at least 8 weeks in duration. In some embodiments, a treatment period is up to about 4 weeks in duration, or up to about 6, 8, 10, 12, 16, 20, 24, 28, 32, 36, 40, 44, or 48 weeks in duration. In some embodiments, a treatment period is up to about 8 weeks in duration. In some embodiments, a treatment period is up to about 12 weeks in duration.

For a subject receiving a dose reduction, in some embodiments, a treatment period at a first dose is paused or stopped prior to starting a subsequent treatment period at a second lower dose. For example, in some embodiments, a first treatment period (e.g., at a dose of 180 mcg per week) is paused or stopped for a period of at least 1 week, 2 weeks, 3 weeks, 4 weeks or longer prior to starting a second treatment period (e.g., at a dose of 120 mcg per week).

In some embodiments, a subject is administered a first dose of 180 micrograms QW for at least 8 weeks before there is a dose reduction. In some embodiments, a subject is administered a first dose of 180 micrograms QW for at least 8-12 weeks before there is a dose reduction.

In some embodiments, if the subject has an absolute neutrophil count (ANC) of between ≥ to 500/mm³ and < 750/mm³, or between ≥ to 400/mm³ and < 650/mm³, or between ≥ to 400/mm³ and < 850/mm³, the subject will begin the second treatment period.

In some embodiments, if the subject has an ANC of < 500/mm³, dosing of the subject will stop until the subject has an ANC of > 1000/mm³ and then dosing will be resumed for a second treatment period. In another embodiment, if the subject has an ANC of < 400/mm³, dosing of the subject will stop until the subject has an ANC of > 750/mm³ and then dosing will be resumed for a second treatment period.

In some embodiments, if the subject has a platelet level of <50,000 then subject will begin the second treatment period or if a subject has a platelet level of <25,000 then subject will discontinue treatment.

In some embodiments, if the subject has a total bilirubin (TBILI) > 2.5 x upper limit of the normal range (ULN) and direct bilirubin (DB) > 3 x ULN, dosing of the subject will stop until the subject has a TBILI ≤1.5 x ULN and then dosing will resume for a second treatment period.

In some embodiments, if the subject has a TBILI > 3 x ULN and DB > 3 x ULN, dosing of the subject will be interrupted until the TBILI ≤1.5 x ULN and then dosing will resume for a second treatment period.

In some embodiments, if the subject has an ALT (or AST) ≥ 20 × ULN and TBILI and/or international normalized ratio (INR) < Grade 2, dosing of the subject will be interrupted until the ALT/AST <10XULN and then dosing will resume for a second treatment period. In some embodiments, if the subject has an absolute neutrophil count (ANC) of alanine aminotransferase (ALT) (or aspartate aminotransferase (AST)) ≥ 20 × ULN and TBILI and/or INR < Grade 2 for a second time, dosing of the subject will be interrupted and then dosing will resume for a second treatment period.

In some embodiments. if the subject has an ALT (or AST) ≥ 15 - 20 × ULN and TBILI and/or INR < Grade 2, dosing of the subject will be interrupted dosing until the ALT/AST <10XULN and then dosing will resume for a second treatment period; or if the subject has an ANC of ALT (or AST) ≥ 15 - 20 × ULN and TBILI and/or INR < Grade 2 for a second time, dosing of the subject will interrupt dosing until the ALT/AST <10XULN and then dosing will resume for a second treatment period.

In some embodiments, the dose resumption after an interruption or stopping is resumed one week, two weeks, three weeks or four weeks after the interruption on stopping.

In some embodiments, if the subject has an ALT (or AST) ≥ 15 × ULN and TBILI and/or INR < Grade 2, dosing of the subject will be interrupted until the ALT/AST <10XULN and then dosing will resume for a second treatment period. In some embodiments, if the subject has an ANC of ALT (or AST) ≥ 15 × ULN and TBILI and/or INR < Grade 2 for a second time, dosing of the subject will be interrupted and then dosing will resume for a second treatment period.

In some embodiments, if the subject has an ALT (or AST) ≥ 5×ULN and TBILI and/or INR ≥ Grade 2, treatment of the subject will terminate.

In some embodiments, if the subject has an ALT (or AST) ≥ 10×ULN and TBILI and/or INR ≥ Grade 3, treatment of the subject will terminate.

In some embodiments, if the subject experiences an adverse event ≥ Grade 3, dosing of the subject will stop until the event resolves or is ≤ a Grade 1 and the dosing will resume for a second treatment period.

In some embodiments, if the subject experiences a second adverse event of ≥ Grade 3, dosing of the subject will be interrupted and then resume dosing for a third treatment period.

In some embodiments, if a subject has a creatinine clearance level of < 50 mL/min, treatment of the subject is discontinued.

In certain embodiments, subjects with a 4× increase in baseline GGT, ALT/AST or alkaline phosphatases or > Bili 1.5 mg/dL, direct Bilirubin >0.6 (if Gilbert Syndrome is present) during any treatment period, may be prescribed ursodeoxycholic acid for “liver protection”.

In certain embodiments, the subject is also administered remdesivir, chloroquine, tenofovir, entecavir, protease inhibitors (lopinavir/ritonavir) for treatment of the coronavirus.

In certain embodiments, the subject is also administered annexin-5, anti-PS monoclonal or polyclonal antibodies, bavituximab, and/or bind to viral glucocorticoid response elements (GREs), retinazone and RU486 or derivatives, cell entry inhibitors, uncoating inhibitors, reverse transcriptase inhibitors, integrase inhibitors, transcription inhibitors, antisense translation inhibitors, ribozyme translation inhibitors, prein processing and targeting inhibitors, protease inhibitors, assembly inhibitors, release phase inhibitors, immunosystem modulators and vaccines, including, but not limited to Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer, Tea tree oil, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, and combinations thereof.

### Duration of Treatment and Treatment Endpoints

Subjects may receive interferon lambda therapy for a predetermined time, an indefinite time, or until an endpoint is reached. Treatment may be continued for at least two to three weeks, or from one to 12 weeks. In some embodiments, therapy is administered weekly for at least 30 days, at least 60 days, at least 90 days, at least 120 days, at least 150 days, or at least 180 days. In some embodiments, weekly treatment is continued for at least 2 months, at least 3 months, at least 4 months, at least 5 months, at least 6 months, at least 7 months, at least 8 months, at least 9 months, at least 10 months, at least 11 months, at least one year, at least 15 months, at least 18 months, or at least 2 years. In some embodiments, therapy is for at least 6 weeks, 12 weeks, 18 weeks, 24 weeks, 30 weeks, 36 weeks, 42 weeks, 48 weeks, 60 weeks, 72 weeks, 84 weeks, or 96 weeks. In other embodiments, treatment is continued for the rest of the subject’s life or until administration is no longer effective in maintaining the virus at a sufficiently low level to provide meaningful therapeutic benefit.

In accordance with the methods herein, some subjects with COVID-19 infection will respond to therapy as described herein by clearing virus to undetectable levels. In some embodiments, for subjects in which coronavirus RNA levels are below the level of detection, treatment is suspended unless and until the coronavirus levels return to detectable levels. Other subjects will experience a reduction in viral load and improvement of symptoms but will not clear the virus to undetectable levels but may remain on therapy for a defined period of time or so long as it provides therapeutic benefit.

In some embodiments, treatment with interferon lambda therapy results in a reduction of coronavirus viral load in the subject of at least 1.5 log10 coronavirus RNA copies/mL serum when measured after 48 weeks of treatment. In some embodiments, treatment with interferon lambda therapy results in a reduction of coronavirus viral load in the subject of at least 2.0 log10 coronavirus RNA copies/mL serum when measured after 48 weeks of treatment. In some embodiments, treatment with interferon lambda therapy results in a reduction of coronavirus viral load in the subject of at least 2.5 log10 coronavirus RNA copies/mL serum when measured after 48 weeks of treatment.

In some embodiments, treatment with interferon lambda therapy results in a sustained reduction of coronavirus viral load (e.g., a decrease of at least 1.5 log10 coronavirus RNA IU/mL serum, at least 2.0 log10 coronavirus RNA copies/mL serum or at least 2.5 log10 coronavirus RNA IU/mL serum, or a decrease in coronavirus RNA to undetectable levels) that is sustained for a period of time (e.g., 1 month, 3 months, 6 months) while the course of treatment is still ongoing.

In some embodiments, treatment with interferon lambda therapy results in a sustained reduction of coronavirus viral load, e.g., reduction of coronavirus viral load, that is sustained for a period of time (e.g., 1 month, 3 months, 6 months, 1 year or longer) or until re-infection occurs or forever, after the course of treatment is finished.

As used herein, duration of viral shedding may be, for example, determined by RT-PCR negativity. The duration of viral shedding may be determined, for example, by clinical improvement O2 status. In some embodiments, the rate or amount of viral shedding is determined by RT-PCR negativity or measurement of a reduced amount of virus (e.g., a reduced viral load).

In some embodiments, treatment with inteferon lambda therapy results in the production of antibodies against SAR-CoV-2 in the subject. In some embodiments, treatment with inteferon lambda therapy increases the quantity of SAR-CoV-2 antibodies in the subject.

In certain embodiments, the subjects have mild disease, and non-hospitalized; have mild to moderate disease, and non-hospitalized; have mild to moderate disease and are hospitalized; have mild to moderate disease, are hospitalized and requiring supportive O₂; exposed to SARS-CoV-2 with no symptoms. For example, the subject may receive a dose of 120 or 180 mcg weekly subcutaneous injection of interferon lambda.

In certain embodiments, the subjects have mild to moderate disease, are hospitalized and will be administered one or two doses of 120 or 180 mcg weekly subcutaneous injection of interferon lambda. In these embodiments, RT-PCR may be used to test for viral load on one or more of the days following administration (e.g., at days 7 and 14 after administration). Subjects receiving one or two administrations of interferon lambda may exhibits lower viral loads than those patients with similar disease status at initiation of treatment that received only supportive care.

In one embodiment, the subject with mild to moderate disease receives one or two administrations of lambda. In this embodiment, the subject may exhibit a lower level or duration of viral shedding (i.e. as compared to non-treated patients).

In one embodiment, subjects that are hospitalized and require supportive oxygen, are administered two doses of interferon lambda, such doses being administered a week apart. In this embodiment, the subject may demonstrate a clinical improvement in oxygen status (for example measured on an ordinal scale) as compared to subjects with similar disease status at initiation of treatment only receiving standard of care.

In one embodiment, subjects with mild to moderate disease and either non-hospitalized or hospitalized, receive two (2) doses of 120 or 180 mcg weekly subcutaneous injection of interferon lambda. In this embodiment, the subjects may have a lower rate of viral shedding as measured by RT-PCR negativity on one or more of the days following administration (e.g., at Day 7 and/or Day 14).

In some aspects, this disclosure provides a method of preventing an infection with SARS-CoV-2 in non-hospitalized subjects. According to one embodiment, two (2) doses of 120 or 180 mcg weekly subcutaneous injection of interferon lambda are administered to the subject.

In some aspects, the disclosure provides a method of preventing an infection with SARS-CoV-2 in subjects who have been exposed to SARS-CoV-2. In one embodiment, the subject receives one interferon lambda 180 mcg subcutaneous injection. In one embodiment, the subject then receives an RT-PCR test for viral load to determine if infection has happened. In one embodiment, subjects have lower conversion rate to infection than those that do not receive a lambda injection. In one embodiment, the subject is a subject that has had exposure with no confirmed infection. The subject has a decrease in conversion as compared to a subject that was exposed, had no treatment and resulted in a confirmed infection (control group).

In some aspects, the disclosure provides a method of treating an infection with SARS-CoV-2 in subjects having a confirmed SARS-CoV-2 infection. In one embodiment, the subject has confirmed mild COVID-19 infection with uncomplicated disease. In one embodiment, the subject is administered interferon lambda 180 mcg subcutaneous injection per week for two weeks. In one embodiment the subject is administered a single interferon lambda 180 mcg subcutaneous injection.

In one embodiment, interferon lambda 180 mcg is administered to a subject, wherein the subject has one or more of the following, as compared to a control: a reduced duration of viral shedding of SARS-CoV-2 virus, a reduction in the duration of symptoms; and a reduction in the rate of hospitalization following administration (e.g., reduced hospitalization between Day 1 and Day 28 of treatment). In one embodiment, the subject has mild COVID-19. In one embodiment, the subject has mild to moderate COVID-19.

### Antiviral Co-Therapy

In some embodiments, a subject who is administered interferon lambda therapy according to the present disclosure may also be treated with one or more other antiviral agents, and other agents.

In some embodiments, a subject who is administered interferon lambda therapy is treated with an antiviral agent that is used for the treatment of other viruses.

In some embodiments, interferon lambda can be formulated into a preparation for injection by dissolving, suspending or emulsifying the interferon lambda in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Unit dosage forms for injection or intravenous administration may comprise in a composition as a solution in sterile water, normal saline or another pharmaceutically acceptable carrier. Appropriate amounts of the active pharmaceutical ingredient for unit dose forms of interferon lambda are provided herein.

In some embodiments, interferon lambda (e.g., an interferon lambda 1 such as interferon lambda 1a) or an analog thereof is formulated and/or administered and/or modified as described in any of U.S. Pat. Nos. 6,927,040, 7,038,032, 7,135,170, 7,157,559, and 8,980,245, US 2009/0326204, US 2010/0222266, US 2011/0172170, and US 2012/0036590, each of which is incorporated by reference herein in their entireties.

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a method of treating a coronavirus infection in a subject, the method comprising subcutaneously administering to the subject a therapeutically effective amount of pegylated interferon lambda-1a until one or more of: a sustained reduction of coronavirus viral load is reached, a decrease in coronavirus RNA to undetectable levels is reached, a decrease in a rate or an amount of viral shedding is reached, or an improvement in the subject’s symptoms is reached.

Embodiment 2 is the method of embodiment(s) 1, wherein the pegylated interferon lambda-1a is administered for at least 1 week, 2 weeks, 3 weeks, 4 weeks, from 1-12 weeks, from 2 - 12 weeks, or from between 3 weeks and 24 weeks.

Embodiment 3 is the method of embodiment(s) 1 or 2, wherein the pegylated interferon lambda-1a is administered at a dose of 180 micrograms once a week, 90 micrograms twice per week, 80 micrograms twice per week, or 180 micrograms per week.

Embodiment 4 is the method of embodiment(s) 1 or 2, wherein the pegylated interferon lambda-1a is administered at a dose of 120 micrograms once a week, 60 micrograms twice per week, 70 micrograms twice per week, or 120 micrograms per week.

Embodiment 5 is the method of embodiment(s) 1 or 2, wherein the method comprises administering (i) 160 - 180 micrograms pegylated interferon lambda-1a per week for a first treatment period, and then 150 - 170 micrograms per week for a second treatment period; or (ii) 180 micrograms per week for a first treatment period, and then between 170 - 120 micrograms per week for a second treatment period, wherein the doses for each of (i) and (ii) may be divided into more than one dose per week.

Embodiment 6 is the method of embodiment(s) 1 or 2, wherein the method comprises administering the pegylated interferon lambda-1a at a dose of 120 micrograms per week for a first treatment period, and then at a dose of 80 micrograms per week for a second treatment period; or at a dose of 180 - 120 micrograms per week for a first treatment period and then at a dose of 120 -80 micrograms per week for a second treatment period, wherein the doses may be divided into more than one dose per week.

Embodiment 7 is the method of embodiment(s) 5 or 6, wherein the first treatment period is longer than the second treatment period, or the second treatment period is longer than the first treatment period, or first treatment period and the second treatment period are the same length of time.

Embodiment 8 is the method of any of embodiment(s) 5 to 7, wherein the first treatment period has a duration of at least 1 week, at least 2 weeks, at least 6 weeks, or at least 8 weeks.

Embodiment 9 is the method of any of embodiment(s) 1 to 8, wherein treatment results in a reduction of coronavirus viral load in the subject of at least 2.0 log10 coronavirus RNA IU/mL serum.

Embodiment 10 is the method of any of embodiment(s) 1 to 9, wherein treatment results in an improvement in the subject’s symptoms.

Embodiment 11 is the method of embodiment(s) 1 to 10, wherein the improvement in a subject’s symptoms include a reduction in fever, feeling less tired, a decrease in coughs, less or no shortness of breath, decreased feeling of aches and pains, and less or no diarrhea.

Embodiment 12 is the method of any of embodiment(s) 1 to 11, wherein treatment results in a coronavirus viral load that is below the level of detection.

Embodiment 13 is the method of any of embodiment(s) 1 to 12, wherein the method further comprises administering to the subject an antiviral.

Embodiment 14 is the method of embodiment(s) 13, wherein the antiviral comprises one or more of remdesivir, chloroquine, tenofovir, entecavir, protease inhibitors (lopinavir/ritonavir).

Embodiment 15 is the method of any of embodiment(s) 1 to 14, wherein prior to treatment, the subject has a baseline viral load of up to about 10⁴ coronavirus RNA copies per mL sample.

Embodiment 16 is the method of any of embodiment(s) 1 to 15, wherein a durable virologic response (DVR) is seen in the subject after administration.

Embodiment 17 is the method of any of embodiment(s) 1 to 16, where the subject has one or more of the following symptoms: pneumonia, fever, cough, shortness of breath, and muscle ache. Other symptoms my include confusion, headache and sore throat.

Embodiment 18 is the method of any of embodiment(s) 1 to 17, wherein the coronavirus is a zoonotic virus.

Embodiment 19 is the method of any of embodiment(s) 1 to 18, wherein the pegylated interferon lambda-1a is administered during an early phase of the coronavirus infection, and wherein the treatment shortens the duration of the coronavirus infection and prevents development of respiratory complications.

Embodiment 20 is the method of embodiment(s) 19, wherein the early phase of the coronavirus infection comprises one or more of: days 1-10 after initial viral load is determined, prior to experiencing respiratory symptoms that require hospitalization; a period when the subject is experiencing mild to moderate respiratory symptoms; a period when the subject is asymptomatic; or a period when the subject displays mild symptoms of respiratory infection with no respiratory distress.

Embodiment 21 is the method of embodiment(s) 20, wherein the mild symptoms of respiratory infection with no respiratory distress comprises a temperature <39.0° C., respiratory rate < 25, O₂% Sat > 95% in room air or with supplemental oxygen through nasal cannula, or P/F ratio > 150.

Embodiment 22 is the method of any of embodiment(s) 1 to 21, wherein the subject has not demonstrated one or more of the following abnormal laboratory test in the 12 months prior to administration: platelet count <90,000 cells/mm3; white blood cell (WBC) count <3,000 cells/mm3; absolute neutrophil count (ANC) <1,500 cells/mm3; hemoglobin <11 g/dL for women and <12 g/dL for men; estimated creatinine clearance (CrCl) < 50 mL/min by Cockroft-Gault formulation; ALT and/or ALT levels > 10 times the upper limit of normal; bilirubin level ≥ 2.5 mg/dL unless due to Gilbert’s syndrome; serum albumin level <3.5 g/dL; or international normalized ratio (INR) ≥1.5 (except patients maintained on anticoagulant medications).

Embodiment 23 is the method of any of embodiment(s) 1 to 22, wherein the rate or amount of viral shedding is determined by RT-PCR negativity or a measurement of a reduced amount of virus.

Embodiment 24 is the method of any of embodiment(s) 1 to 23, wherein an improvement in symptoms is determined by clinical improvement O2 status.

Embodiment 25 is the method of any of embodiment(s) 1 to 24, wherein the subject is a mild, non-hospitalized subject; a mild to moderate, non-hospitalized subject; a mild to moderate, hospitalized subject; a mild to moderate, hospitalized and requiring supportive O2 subject; or an exposed subject with no symptoms.

Embodiment 26 is the method of any of embodiment(s) 1 to 25, wherein the pegylated interferon lambda-1a is administered at a dose of 120 or 180 mcg weekly.

Embodiment 27 is the method of any of embodiment(s) 1 to 26, wherein the subject is a mild to moderate, hospitalized subject, and wherein the pegylated interferon lambda-1a is administered as one or two doses of 120 or 180 mcg weekly.

Embodiment 28 is the method of any of embodiment(s) 1 to 27, wherein RT-PCR is used to test for viral load at days 7 and 14 of treatment, and wherein the subject exhibits lower viral loads at days 7 and 14 than a patient with a similar disease status at initiation of treatment that received only standard supportive care.

Embodiment 29 is the method of any of embodiment(s) 1 to 28, wherein the subject is a mild to moderate subject, and wherein the subject exhibits a decreased rate or amount of viral shedding.

Embodiment 30 is the method of any of embodiment(s) 1 to 29, wherein the subject is a mild to moderate, hospitalized subject requiring supportive oxygen, and wherein the subject demonstrates clinical improvement in oxygen status (ordinal scale) as compared to a patient with similar disease status at initiation of treatment that only received standard supportive care.

Embodiment 31 is the method of embodiment 30, wherein the subject is administered two doses of interferon lambda one week apart.

Embodiment 32 is the method of any of embodiment(s) 1 to 31, wherein the subject has mild to moderate disease, is non-hospitalized or hospitalized, wherein the pegylated interferon lambda-1a is administered at a dose of 120 or 180 mcg twice weekly, and wherein the subject exhibits a lower rate of viral shedding as measured by RT-PCR negativity following first administration of treatment (e.g., a first dose of interferon lambda; e.g., by Day 7 and/or Day 14 of treatment).

Embodiment 33 is a method of preventing or reducing the incidence of infection in a subject with SARS-CoV-2, the method comprising administering to the subject interferon lambda by subcutaneous injection in a dose of 120 or 180 mcg weekly or biweekly, wherein the subject is RT-PCR negative a first dose of interferon lambda (e.g., by Day 7 and/or Day 14 of treatment).

Embodiment 34 is the method of embodiment(s) 33, wherein the subject has a lower RT-PCR level of SARS-CoV-2 than a subject receiving standard supportive care.

Embodiment 35 is a method of preventing or reducing the incidence of a SARS-CoV-2 infection in a subject exposed to SARS-CoV-2, the method comprising administering to the subject 180 mcg of interferon lambda as a subcutaneous injection, wherein the subject exhibits a lower viral load at day 7 after the injection than a subject with similar disease status at initiation of treatment receiving standard supportive care.

Embodiment 36 is the method of embodiment(s) 35, wherein the subject exhibits a lower conversion rate to infection than a patient with similar disease status at initiation of treatment that was not administered interferon lambda.

Embodiment 37 is the method of any of embodiment(s) 35 to 36, wherein the subject has had exposure to SARS-CoV-2 with no confirmed infection.

Embodiment 38 is a method of treating a subject having a SARS-CoV-2 infection or having been exposed to SARS-CoV-2, the method comprising administering to the subject interferon lambda at a dose of 180 mcg, wherein the subject has one or more of the following, as compared to a control: a reduced duration of viral shedding of SARS-CoV-2 virus, a reduction in the duration of symptoms, or a reduction in the rate of hospitalization following first administration of treatment (e.g., a first dose of interferon lambda; e.g., between Day 1 and Day 28 of treatment).

Embodiment 39 is the method of embodiment 38, wherein the interferon lambda is administered subcutaneously.

Embodiment 40 is the method of any of embodiment(s) 38 to 39, wherein the interferon lambda is interferon lambda-1a.

Embodiment 41 is the method of any of embodiment(s) 38 to 40, wherein the interferon lambda is pegylated interferon lambda.

Embodiment 42 is the method of any of embodiments(s) 38 to 41, wherein the rate of hospitalization includes visits to an emergency room.

Embodiment 43 is the method of any of embodiment(s) 1 to 42, wherein the subject has a viral load equal to or greater than 6 log₁₀ copies/mL.

Embodiment 44 is the method of any of embodiment(s) 1 to 43, wherein the subject has a viral load of from about 6 log₁₀ IU/mL to about 11 log₁₀ IU/mL.

Embodiment 45 a method of treating a coronavirus infection in a subject, the method comprising subcutaneously administering to the subject from 120 mcg to 180 mcg of interferon lambda, wherein the subject has a viral load greater than or equal to 10⁶ SARS-CoV2 RNA copies/mL or greater than or equal to 6 log₁₀ IU/mL.

Embodiment 46 is the method of embodiment 45, wherein the interferon lambda is administered at a dose of 120 mcg or 180 mcg, and wherein the subject exhibits a lower rate of viral shedding as measured by viral load negativity at Day 7, Day 14, and/or Day 28 of treatment as compared to at the initiation of treatment.

Embodiment 47 is the method of embodiment(s) 45-46, wherein the subject has a viral load of from about 6 log₁₀ IU/mL to about 11 log₁₀ IU/mL.

Embodiment 48 is the method of embodiment 1 or embodiment 45, wherein the time to shedding cessation is faster in a seropositive subject relative to a seronegative subject at baseline.

Embodiment 49 is the method of any of embodiment(s) 45-48, wherein the subject has a greater decline in SARS-CoV-2 RNA viral load decline from baseline at Day 5 of treatment, as compared to a control.

Embodiment 50 is the method of any of embodiment(s) 45-49, wherein the subject is about 4.1-fold or 95% more likely to clear virus by Day 7 of treatment, as compared to a control.

Embodiment 51 is the method of any of embodiment(s) 44-50, wherein the subject has a viral load greater than or equal to 6 log₁₀ IU/mL, and wherein the subject is viral negative by Day 7 of treatment.

Embodiment 52 is the method of any of embodiment(s) 44-51, wherein the subject clears the virus by Day 7 of treatment.

Embodiment 53 is the method of any of embodiment(s) 44-52, wherein the interferon lambda is pegylated interferon lambda-1a.

### EXAMPLES

The following examples are provided to illustrate, but not to limit, the claimed invention.

### Example 1. Clinical Study Protocol for Treating Coronavirus Subjects With Pegylated Interferon Lambda

The incubation period is estimated at ^{~}5 days (95% confidence interval, 4 to 7 days). Frequently reported signs and symptoms include fever (83-98%), cough (76%-82%), and myalgia or fatigue (11-44%) at illness onset. Sore throat has also been reported in some patients early in the clinical course. Less commonly reported symptoms include sputum production, headache, hemoptysis, and diarrhea. The fever course among patients with SARS-CoV-2 infection is not fully understood; it may be prolonged and intermittent. Asymptomatic infection has been described in one child with confirmed SARS-CoV-2 infection and chest computed tomography (CT) abnormalities.

Risk factors for severe illness may include for older patients and those with chronic medical conditions may be at higher risk for severe illness. Nearly all reported cases have occurred in adults (median age 59 years). In one study of 425 patients with pneumonia and confirmed SARS-CoV-2 infection, 57% were male. Approximately one-third to one-half of reported patients had underlying medical comorbidities, including diabetes, hypertension, and cardiovascular disease.

This example describes a clinical study protocol for evaluating the safety, tolerability, and pharmacodynamics of pegylated interferon lambda monotherapy in subjects with chronic Coronavirus infection.

Protocol Synopsis

| | |
|---|---|
| Product | Pegylated interferon lambda-1a (PEG-IFN-λ) |
| Study design | Randomized, open-label study of interferon lambda 120 or 180 µg subcutaneous (SC) injection weekly for 12 weeks (or fewer if virus is no longer detectable and subject no longer has symptoms of infection) in subjects with acute or chronic Coronavirus infection. Clinic visits at baseline (Day 1), every week until Week 12. PD/efficacy of interferon lambda will be assessed by measuring coronavirus viral loads, viral serologies. Safety and tolerability of interferon lambda will be assessed by AE monitoring, clinical laboratory tests, physical examinations, vital signs, body weight, and concomitant medications. All enrolled subjects will be followed for an additional 12 weeks off-treatment. All monthly follow-up visits will include evaluations of viral load (coronavirus), and all of the safety measures listed above. |
| Test product, dose, and method of administration | Pegylated interferon lambda-1a (PEG-IFN-λ) (Lambda), 120 or 180 µg, |
| Evaluation | Proportion of subjects with undetectable coronavirus RNA 12 weeks after EOT (SVR-12) |
| | Change from baseline in coronavirus viral load at Week 12 or when stop treatment due to symptomatic improvement (EOT) |
| | Additional PD/efficacy endpoints include: |
| | Proportion of subjects with undetectable coronavirus RNA 12 weeks after EOT (SVR-24) |
| | Change from baseline in coronavirus viral load |
| | Safety endpoints include: |
| | Treatment-emergent AEs and SAEs |
| | Treatment-emergent treatment-related AEs and SAEs |
| | AEs leading to early discontinuation of study treatment |
| | AEs leading to dose reduction |
| | Treatment-emergent changes in clinical laboratory findings |
| | Treatment-emergent changes in vital signs |
| | Treatment-emergent changes in ECG findings |
| | Treatment-emergent changes in physical examination results |
| | Usage of concomitant medications during the study |

### Example 2. Detection Methods of Coronavirus

One method to detect SARS-CoV-2 is by using the Real-Time RT-PCR Panel for Detection 2019-Novel Coronavirus, by the Centers for Disease Control and Prevention, Respiratory Viruses Branch, Division of Viral Diseases. The publication of this method is hereby incorporated by reference.

Primers and Probes that may be used to detect SARS-CoV-2 are described below. For example, Table 1 provides exemplary primer sequences, which are identified herein as SEQ ID NOs: 2-13 (from top row to bottom row).

TABLE 1

SARS-CoV-2 Real-Time rRT-PCR Panel Primers and Probes

| | | | | |
|---|---|---|---|---|
| Name | Description | Oligonucleotide Sequence (5′->3′) | Label¹ | Working Conc. |
| 2019-nCoV_N1-F | 2019-nCoV_N1 Forward Primer | 5′-GAC CCC AAA ATC AGC GAA AT-3′ | None | 20 µM |
| 2019-nCoV_N1-R | 2019-nCoV_N1 Reverse Primer | 5′-TCT GGT TAC TGC CAG TTG AAT CTG-3′ | None | 20 µM |
| 2019-nCoV_N1-P | 2019-nCoV_N1 Probe | 5′-FAM-ACC CCG CAT TAC GTT TGG TGG ACC-BHQ1-3′ | FAM, BHQ-1 | 5 µM |
| 2019-nCoV_N2-F | 2019-nCoV_N2 Forward Primer | 5′-TTA CAA ACA TTG GCC GCA AA-3′ | None | 20 µM |
| 2019-nCoV_N2-R | 2019-nCoV_N2 Reverse Primer | 5′-GCG CGA CAT TCC GAA GAA-3′ | None | 20 µM |
| 2019-nCoV_N2-P | 2019-nCoV_N2 Probe | 5′-FAM-ACA ATT TGC CCC CAG CGC TTC AG-BHQ1-3′ | FAM, BHQ-1 | 5 µM |
| 2019-nCoV_N3-F | 2019-nCoV_N3 Forward Primer | 5′-GGG AGC CTT GAA TAC ACC AAA A-3′ | None | 20 µM |
| 2019-nCoV_N3-R | 2019-nCoV_N3 Reverse Primer | 5′-TGT AGC ACG ATT GCA GCATTG-3′ | None | 20 µM |
| 2019-nCoV_N3-P | 201.9-nCoV_N3 Probe | 5′-FAM-AYC ACA TTG GCA CCC GCA ATC CTG-BHQ1-3′ | FAM, BHQ-1 | 5 µM |
| RP -F | RNAse P Forward Primer | 5′-AGA TTT GGA CCT GCG AGC G-3′ | None | 20 µM |
| RP-R | RNAse P Reverse Primer | 5′-GAG CGG CTG TCT CCA CAA GT-3′ | None | 20 µM |
| RP-P | RNAse P Probe | 5′-FAM-TTC TGA CCT GAA GGC TCT GCG CG-BHQ-1-3′ | FAM, BHQ-1 | 5 µM |

TaqMan® probes are labeled at the 5′-end with the reporter molecule 6-carboxyfluorescein (FAM) and with the quencher, Black Hole Quencher 1 (BHQ-1) (Biosearch Technologies, Inc., Novato, CA) at the 3′-end.

### Diagnostic Testing of 2019-nCoV (SARS-CoV–2)

Currently, confirmation of SARS-CoV-2 infection (referred to below as 2019-nCoV infection) is performed at CDC using the CDC real-time RT-PCR assay for 2019-nCoVon respiratory specimens (which can include nasopharyngeal or oropharyngeal aspirates or washes, nasopharyngeal or oropharyngeal swabs, broncheoalveolar lavage, tracheal aspirates, or sputum) and serum. Information on specimen collection, handling, and storage is available at: Real-Time RT-PCR Panel for Detection 2019-Novel Coronavirus. After initial confirmation of 2019-nCoVinfection, additional testing of clinical specimens can help inform clinical management, including discharge planning.

### Laboratory and Radiographic Findings: 2019-nCoV(SARS-CoV-2)

The most common laboratory abnormalities reported among hospitalized patients with 2019-nCoVinclude pneumonia on admission, leukopenia (9-25%), leukocytosis (24-30%), lymphopenia (63%), and elevated alanine aminotransferase and aspartate aminotransferase levels (37%). Most patients had normal serum levels of procalcitonin on admission. Chest CT images have shown bilateral involvement in most patients. Multiple areas of consolidation and ground glass opacities are typical findings reported to date.

2019-nCoVRNA has been detected from upper and lower respiratory tract specimens, and the virus has been isolated from bronchoalveolar lavage fluid. The duration of shedding of 2019-nCoV RNA in the upper and lower respiratory tracts is not yet known but may be several weeks or longer, which has been observed in cases of MERS-CoV or SARS-CoV infection.

### Example 3. Clinical Trial

Subjects infected with SARS-CoV-2 will be evaluated for the safety and tolerability of treatment with subcutaneous (S.C.) injections of interferon lambda at doses of 120 or 180 mcg. Subjects will be compared to standard supportive care (control arm) of patients infected with SARS-CoV-2. The study will be a randomized, open label, 2 arm, pilot trial of interferon lambda 180 mcg administered S.C once weekly, for up to two weeks (2 injections at most), in addition to standard supportive care, compared to standard supportive care of up to 2 weeks, in a population of SARS-CoV-2 infected patients.

Patients will be randomized according to 1:1 ratio to one of the trial arms: interferon lambda 180 mcg S.C (intervention arm), or standard care (control arm). Up to 40 patients will be included, each with proven COVID-19 infection by PCR and diagnosed with mild to moderate respiratory symptoms.

Following initial diagnosis of COVID-19, patients will be admitted to a hospital (Day 0). Upon admission, patients will be randomized according to 1:1 ratio to one of the trial arms and receive either interferon lambda 180 mcg S.C (intervention arm) or standard of care (control arm). Patients’ vital signs (temperature, blood pressure, pulse rate per minute, breath rate per minute and oxygen saturation), will be monitored according to standard of care (SoC). Symptom questionnaires will be collected from patients as well as adverse events (AEs) assessment and recording of the need for supportive respiratory measures (SRM) once daily during the period of hospitalization.

Efficacy of interferon lambda will be assessed by PCR analysis for COVID-19 (Fluxergy, Irvine, CA) from respiratory secretions obtained by nasopharyngeal and oropharyngeal swabs, collected consecutively at day 1, 3, 5, 7, 10, 14 and 21 following initial diagnosis or until patients are discharged following achievement of two consecutive PCR negative tests for COVID-19. Safety and tolerability of interferon lambda will be assessed by adverse event (AE) monitoring, vital signs assessment and clinical laboratory tests (complete blood count (CBC), and extended chemistry panel).

Patients will include: Female and male patients over the age of 18; confirmed COVID-19 infection by PCR analysis; hospitalized; and displaying mild to moderate symptoms of respiratory infection, including temperature <39.0° C., respiratory rate < 25, O2 % Sat > 95% in room air or with supplemental oxygen through nasal cannula, P/F ratio > 150).

Patients will be excluded if they have had: treatment with interferons (IFNs) immunomodulators and/or immunosuppressive or B-cell depleting medications within 12 months before screening; previous use of Interferon Lambda; history or evidence of any intolerance or hypersensitivity to IFNs; respiratory infection requiring invasive or non-invasive ventilatory support (bipap or intubation and mechanical ventilation); participation in a clinical trial with use of any investigational drug within 30 days before screening; or history of any of the following diseases or conditions: advanced or decompensated liver disease (presence or history of bleeding varices, ascites, encephalopathy or hepato-renal syndrome); immunologically mediated disease (e.g., rheumatoid arthritis, inflammatory bowel disease, severe psoriasis, systemic lupus erythematosus) that requires more than intermittent nonsteroidal anti-inflammatory medications for management or that requires use of systemic corticosteroids in the 6 months before screening (inhaled asthma medications are allowed); retinal disorder or clinically relevant ophthalmic disorder; any malignancy within 5 years before screening.

Exceptions are superficial dermatologic malignancies (e.g., squamous cell or basal cell skin cancer treated with curative intent); cardiomyopathy, significant ischemic cardiac or cerebrovascular disease (including history of angina, myocardial infarction, or interventional procedure for coronary artery disease), or cardiac rhythm disorder; chronic pulmonary disease (e.g., chronic obstructive pulmonary disease) associated with functional impairment; pancreatitis; severe or uncontrolled psychiatric disorder; active seizure disorder defined by either an untreated seizure disorder or continued seizure activity within the preceding year despite treatment with anti-seizure medication; bone marrow or solid organ transplantation; or any of the following abnormal laboratory test in the 12 months prior to enrollment: platelet count <90,000 cells/mm³; white blood cell (WBC) count <3,000 cells/mm³; absolute neutrophil count (ANC) <1,500 cells/mm³; hemoglobin <11 g/dL for women and <12 g/dL for men; estimated creatinine clearance (CrCl) < 50 mL/min by Cockroft-Gault formulation; ALT and/or ALT levels > 10 times the upper limit of normal; bilirubin level ≥ 2.5 mg/dL unless due to Gilbert’s syndrome; serum albumin level <3.5 g/dL; international normalized ratio (INR) ≥1.5 (except patients maintained on anticoagulant medications).

Efficacy endpoints include: the duration of viral shedding in days since initial diagnosis, as determined by RT-PCR to COVID-19; comparison of time to clinical recovery (TTCR) between interferon lambda and standard care arms; TTCR is defined as the time (in hours) from initiation of trial treatment (interferon lambda or standard care) until normalization of fever, respiratory rate, and oxygen saturation, and alleviation of cough, sustained for at least 72 hours; normalization and alleviation criteria: fever - ≤36.9° C. -axilla or, ≤37.2° C. oral, respiratory rate ≤24/minute in room air, oxygen saturation >94% in room air, cough - mild or absent on a patient reported scale of severe, moderate, mild, absent; comparison of the frequency of requirement for non-invasive or mechanical ventilation between the two treatment arms; comparison of the length of hospital stay between the two treatment arm; comparison of estimated p/f ratio for day of discharge between the study arms; comparison of all-cause mortality at 28 days between the two treatment arms; comparison of the proportion of patients reaching undetectable SARS-CoV-2 levels in respiratory secretions at days 7, 14 and 21 from diagnosis, between the two treatment arms; comparison of the duration of symptoms and signs of respiratory infection associated with COVID-19 between the two treatment arms; comparison of the SARS-CoV-2 viral load in respiratory secretions using a semi-quantitative method between the two treatment arms.

Other endpoints include, for example, the rate of treatment-emergent and treatment-related severe adverse events (SAEs); rate of AEs leading to early discontinuation of trial treatment in patients receiving interferon lambda; comparison of the rate of treatment-emergent changes in clinical laboratory (CBC, liver panel), between the two treatment arms; comparison of the rate of treatment-emergent changes in vital signs and physical examination results between the two treatment arms; and/or usage of concomitant medications during the trial.

The treatment with interferon lambda during early phases of COVID-19 infection shortens the duration of infection and prevents development of respiratory complications.

### Example 4. In Vitro and Animal Analyses

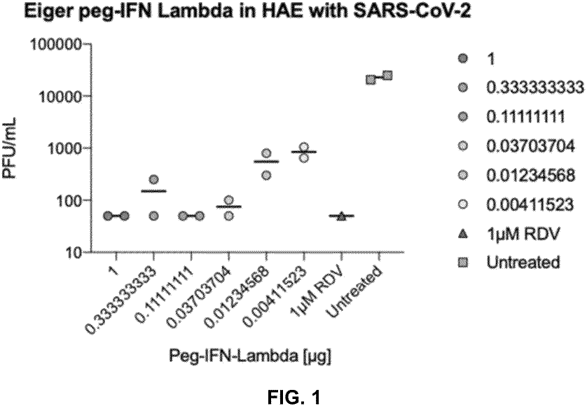

As shown in FIG. 1, primary human airway epithelial cells (donor DD0640p2) were pretreated with interferon lambda in basolateral media for ^{~}24 hr prior to infection. The cells were then infected with 2019-nCoV/USA-WA1/2020 at an MOI 0.5 for 2 hr and washed three times in PBS. After 48 hr, the apical surface was washed with 200 µl to collect secreted virus. Titer was determined via plaque assay in Vero E6 cells. The positive control was 1 µM Remdesivir. This demonstrates the efficacy of interferon lambda for reducing viral load in infected cells.

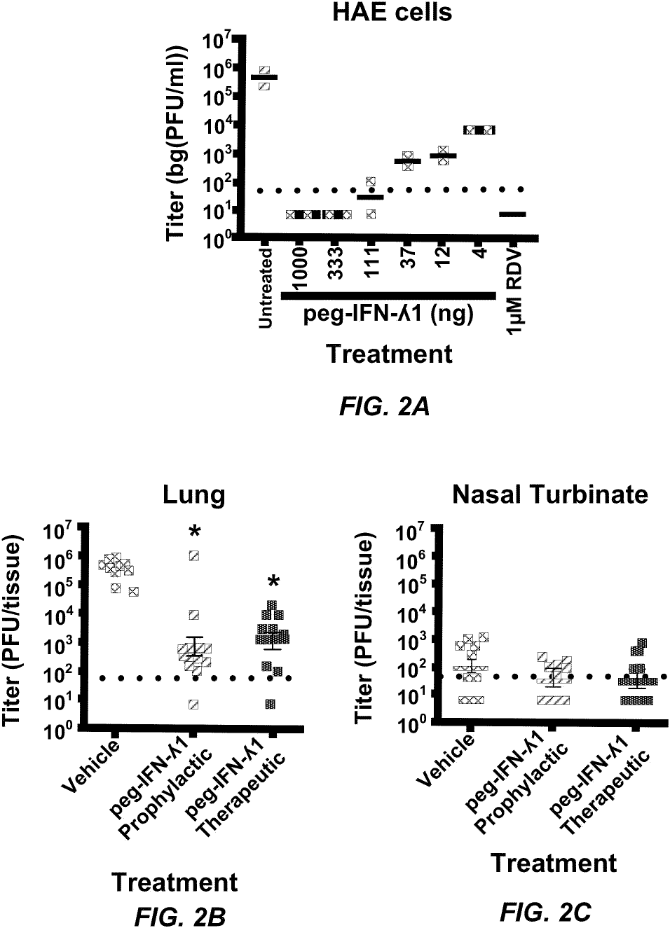

Interferon lambda is a type III interferon whose receptors are largely limited to epithelial cells, including the lungs, liver, and gastrointestinal tract. Treatment with interferons has been employed as pan-viral treatment for several viral infections, including trials for the treatment of SARS-CoV-1 and MERS-CoV infections. Pegylated interferon lambda-1 (peg-IFN-λ1) has been used to treat hepatitis delta virus infection and is studied to treat COVID-19 infection. It was assessed whether peg-IFN-λ1 would initiate an antiviral program capable of inhibiting productive infection of primary human airway epithelial (HAE) cell cultures by SARS-CoV-2. Pretreatment of HAE with peg-IFN-λ1 provided a potent dose dependent reduction in SARS-CoV-2 infectious virus production, as shown in FIG. 2A.

To determine if this in vitro antiviral effect would translate to in vivo efficacy, prophylactic and therapeutic efficacy studies in BALB/c mice were performed. Peg-IFN-λ1 (2 µg) was subcutaneously administered 18 hr prior or 12 hr after infection with 10⁵ pfu SARS-CoV-2 MA. Both prophylactic and therapeutic administration of peg-IFN-λ1 significantly diminished SARS-CoV-2 MA replication in the lung as shown in FIG. 2B. Peg-IFN-λ1 did not alter viral titer in the nasal turbinates as shown in FIG. 2C. This demonstrates that peg-IFN-λ1 exerts potent antiviral activity against SARS-CoV-2 in vitro and can diminish virus replication in vivo when given therapeutically.

### Pegylated-IFN-λ1 Treatment in Vitro

Peg-interferon Lambda-1a was distributed into prefilled syringes, 0.18 mg/syringe (0.4 mg/mL). Primary human airway epithelium cell cultures (HAEs) were grown. Human tracheobronchial epithelial cells were obtained from airway specimens resected from patients undergoing surgery. Primary cells were expanded to generate passage 1 cells and passage 2 cells were plated at a density of 250,000 cells per well supports. HAEs were generated by differentiation at an air-liquid interface for 6 to 8 weeks to form well-differentiated, polarized cultures that resembled in vivo pseudostratified mucociliary epithelium. HAEs were treated with a range of peg-IFN-λ1 doses basolaterally for 24 hrs prior to infection. 1 µM remdesivir was used as a positive control. Cultures were infected at an MOI of 0.5 for 2 hours. Inoculum was removed and culture was washed three times with PBS. At 48 hrs post infection, apical washes were taken to measure viral replication via plaque assays as described above.

### Pegylated-IFN-λ1 Treatment in Vivo

Mice were subcutaneously treated with a single 2 ug dose of peg-IFN-λ1 prophylactically at 18 hrs prior to infection, therapeutically at 12 hrs post infection, or PBS vehicle treated, and infected with 10⁵ plaque forming units (PFU) of SARS-CoV-2 MA intranasally under ketamine/xylazine anesthesia. Body weight was monitored daily. On day 2 post infection, mice were euthanized by isoflurane overdose and tissue samples were harvested for titer analysis as described above.

### Example 5. First Trial Results

Using the methods and criteria described above in Examples 1-3, a first trial was performed. In the first trial, 120 participants were enrolled; 70 (58.3%) were male, 75 (62.5%) identified as Latinx, and the median duration of symptoms prior to randomization was 5 days. Sixty participants were randomly assigned to receive 180 mcg pegylated interferon lambda-1a, and 60 participants were assigned to receive a placebo. At enrollment, 49 (40.8%) participants were SARS-CoV-2 IgG seropositive; seropositive participants had a significantly lower viral load at enrollment compared with seronegative (log₁₀ viral load 2.0 vs. 4.4). Subjects viral samples were taken by oral pharyngeal swabs. The median time to cessation of viral shedding was 7 days in both arms (hazard ratio [HR] for duration of shedding 0.81 comparing Lambda vs. placebo; 95% confidence interval [CI] 0.56 to 1.19; p = 0.29). No difference in time to resolution of symptoms was observed comparing interferon lambda vs. placebo (HR 0.94; 95% CI 0.64 to 1.39; p = 0.76). Two serious adverse events were reported in each arm. Liver transaminase elevations were more common in the interferon lambda vs. placebo arm (15/60 vs 5/60; p = 0.027).

In this study, a single dose of subcutaneous pegylated interferon lambda-1a compared to placebo was well tolerated, but neither shortened the duration of SARS-CoV-2 viral shedding nor improved symptoms.

It was observed that the time to shedding cessation was faster in seropositive subjects (p=0.03). Interferon lambda appeared to hasten shedding cessation among those who were seropositive at baseline and delayed shedding cessation relative to placebo among those who were seronegative at baseline. In the setting of an effective immune response, interferon lambda may augment viral clearance, whereas in the absence of an immune response, lambda protects cells from virus-mediated apoptotic cell lysis.

### Example 6. Second Trial Results

Using the methods and criteria described above in Examples 1-3, a second trial was performed to assess interferon lambda for immediate antiviral therapy at diagnosis in COVID-19 infections. The second trial included a randomized trial of pegylated interferon lambda in outpatients with mild to moderate COVID-19 infection.

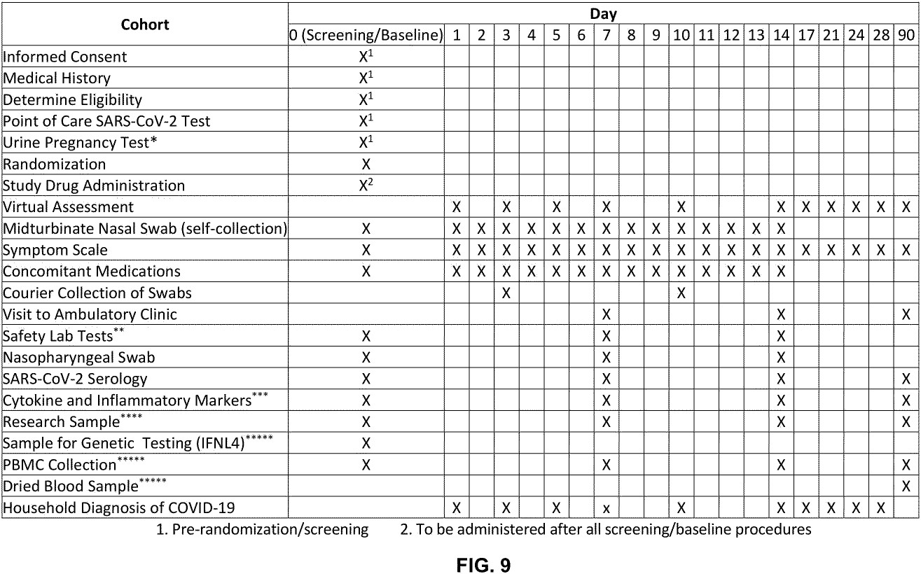

Of 364 individuals approached for the second trial, 105 did not meet inclusion/exclusion criteria and 199 eligible individuals declined to participate as shown in FIG. 9. All 60 randomized individuals received an injection, with one individual lost to follow-up after Day 3. The median age was 46 years (IQR 32-54), 35 (58%) were male and 31 (52%) were Caucasian. Eleven (19%) participants were asymptomatic and the mean time from symptom onset to randomization was 4.5±1.7 days. The median baseline SARS-CoV-2 RNA level was 6.71 (IQR 1.3-8.0) log copies/mL with 10 (33%) individuals in the placebo group and 5 (17%) in the peginterferon-lambda group having undetectable viral load on the day of randomization. Other baseline characteristics were similar between groups (Table 2).

TABLE 2

Baseline Characteristics

| | Peginterferon Lambda n=30 | Placebo n=30 |
|---|---|---|
| Female, n(%) | 18 (60) | 17 (57) |
| Age [years], median (IQR) | 48 (30-53) | 39 (33-55) |

Race/Ethnicity n(%)

| | | |
|---|---|---|
| White | 15 (50) | 16 (53) |
| Black | 1 (3) | 5 (17) |
| Asian | 8 (27) | 7 (23) |
| Other | 6 (20) | 2 (7) |
| Co-morbidity* n(%) | 5 (17) | 4 (13) |
| Body Mass Index, kg/m² mean(sd) | 27.3 (5.2) | 26.1 (4.2) |

Body Mass Index Category, n(%)

| | | |
|---|---|---|
| <25 kg/m² | 9 (30) | 11 (37) |
| 25-30 kg/m² | 15 (50) | 13 (43) |
| >30 kg/m² | 6 (20) | 6 (20) |

Interferon lambda 4 genotype

| | | |
|---|---|---|
| TT | 18 (60) | 16 (57) |
| Non-TT | 12 (40) | 12 (43) |
| Asymptomatic | 5 (17) | 6 (20) |
| Symptom onset to injection (days), mean(sd) | 4.3 (1.7) | 4.7 (1.7) |
| Positive test to injection (days), mean(sd) | 3.2 (1.1) | 3.3 (1.2) |

Baseline laboratory results

| | | |
|---|---|---|
| Hemoglobin (g/L), mean(sd) | 14.7 (1.4) | 14.9 (1.6) |
| White Blood Cells (x10e9/L), mean(sd) | 4.9 (2.1) | 5.1 (1.7) |
| Lymphocytes (x10e9/L), mean(sd) | 1.5 (0.4) | 1.5 (0.5) |
| Neutrophils (x10e9/L), mean(sd) | 2.9 (1.8) | 3.1 (1.6) |
| Platelets (x10e9/L), mean(sd) | 221 (62) | 213 (64) |
| Creatinine (µmol/L), mean(sd) | 80 (14) | 81 (18) |
| Alanine aminotransferase (U/L), mean(sd) | 32 (16) g/dL | 39 (52) g/dL |
| Aspartate aminotransferase (U/L), mean(sd) | 28 (11) g/dL | 32 (24) g/dL |
| Total bilirubin (µmol/L), mean(sd) | 10 (5) g/dL | 12 (10) g/dL |
| SARS-CoV-2 viral load (log copies/mL), mean(sd) | 6.16 (3.14) | 4.87 (3.68) |
| SARS-CoV-2 RNA undetectable at baseline, n(%) | 5 (17) | 10 (33) |
| SARS-CoV-2 RNA ≥ 10E6 copies/mL at baseline, n(%) | 19 (63) | 16 (53) |

* Hypertension, diabetes mellitus, chronic obstructive pulmonary disease, heart disease

Thirty participants were randomly assigned to receive 180 mcg pegylated interferon lambda-1a, and 30 participants were assigned to receive a saline placebo. Patients were followed for 14 days. Nasopharyngeal samples were collected. The baseline SARS-CoV-2 viral load in the interferon lambda group was 6.2 log₁₀ IU/mL and was 4.9 log₁₀ IU/mL in the placebo group. In the interferon lambda group (19 subjects) and placebo (16 subjects) groups, there were a total of 35 subjects with viral loads great or equal to 6 log₁₀ IU/mL. In the interferon lambda group, all subjects were below the level of shedding infectious virus at day 7, had more rapidly cleared the virus than the placebo group, and had a higher probability of clearing the virus at day 7 than the placebo group.

The primary efficacy outcome was the proportion of individuals with a negative MT swab for SARS-CoV-2 at Day 7. The primary safety outcome was the incidence of treatment-emergent severe adverse events by Day 14. Secondary outcomes included: time to SARS-CoV-2 undetectability, change in quantitative SARS-CoV-2 RNA over time, anti-SARS-CoV-2 IgG antibody positivity, the incidence and severity (mild/moderate/severe) of adverse events (AEs), and the proportion hospitalized by Day 14. Detailed directed and open-ended symptoms were assessed serially. Because of overlap between symptoms of COVID-19 and potential peginterferon-lambda AEs, symptoms were recorded and AEs were considered any symptom outside of the directed symptom assessment. Laboratory AE severity was graded using the Common Terminology Criteria for Adverse Events (CTCAE) Version 5.0. An independent Data and Safety Monitoring Committee (DSMC) reviewed safety data after 10, 20 and 30 patients completed 7 days of post-treatment follow-up. After review, the DSMC advised the study team whether to continue enrolment.

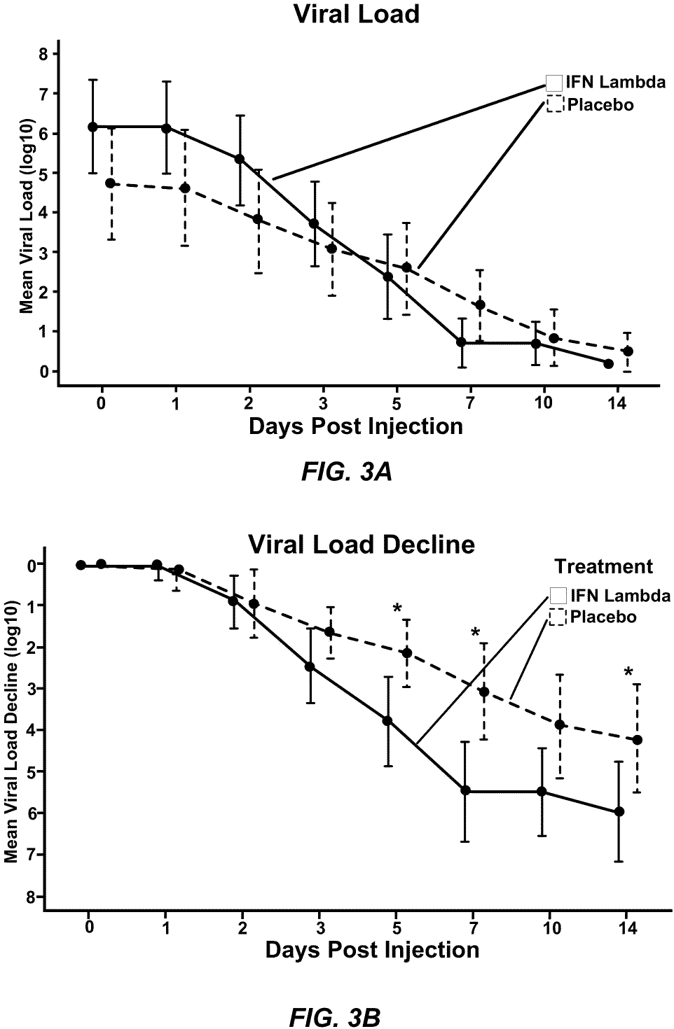

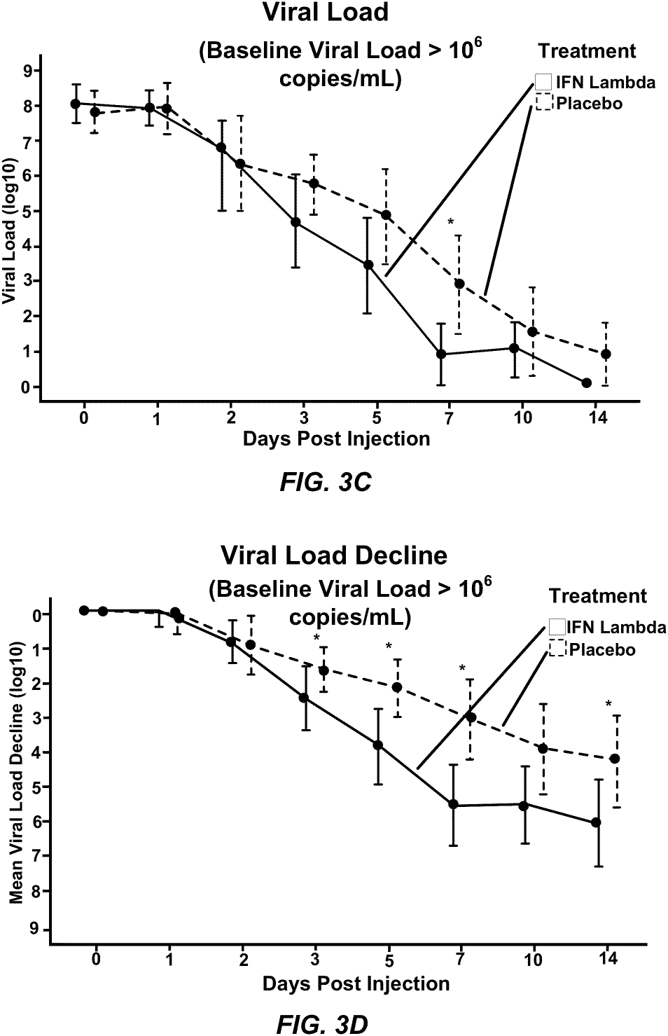

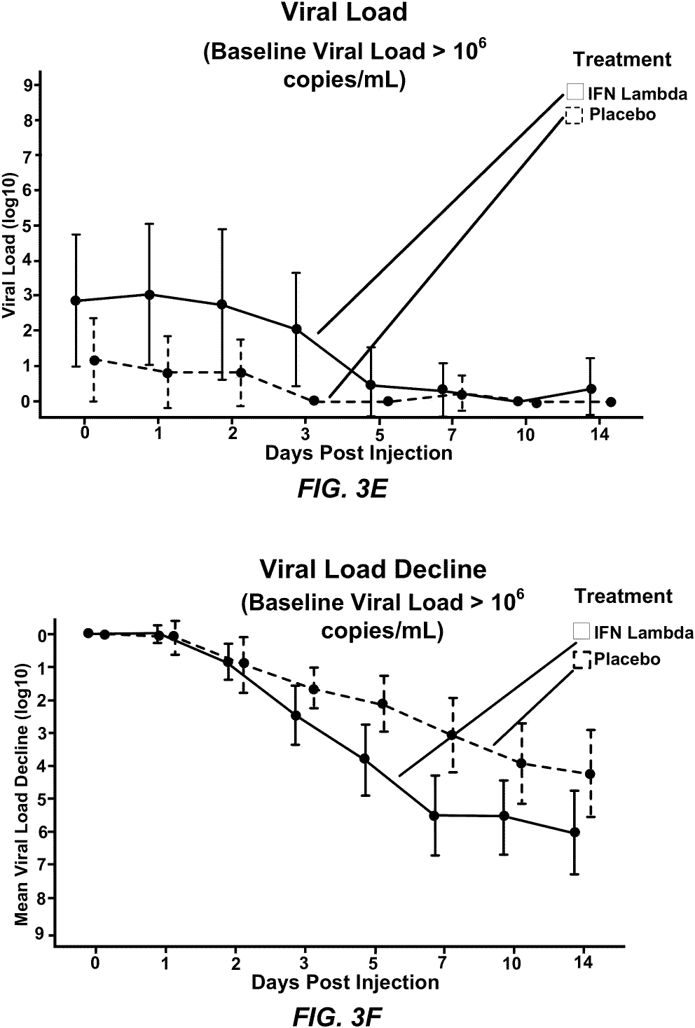

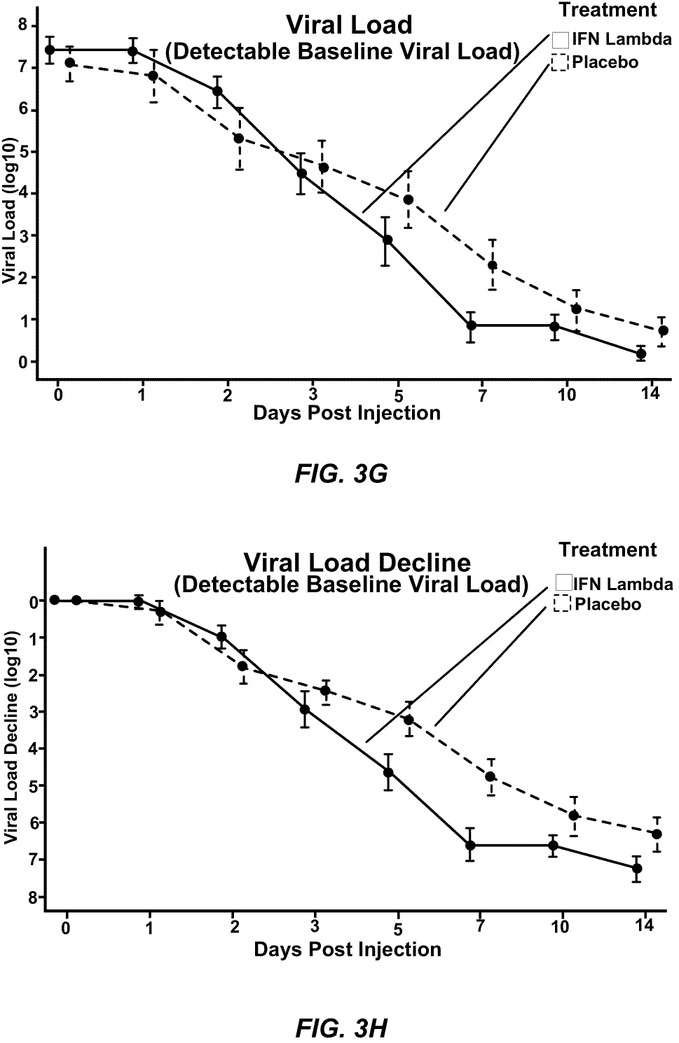

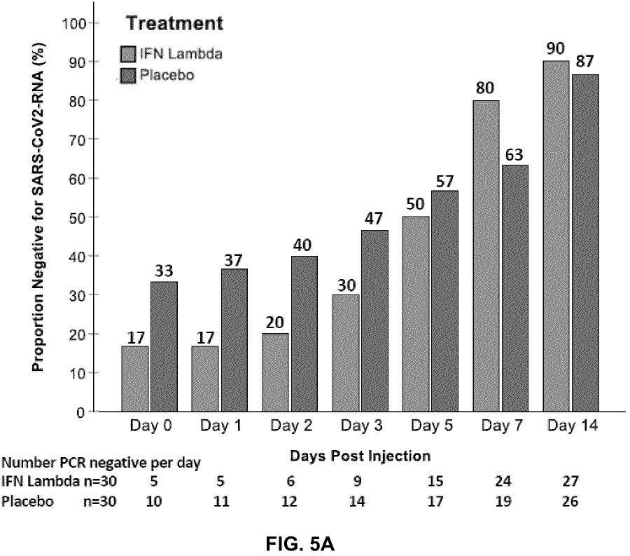

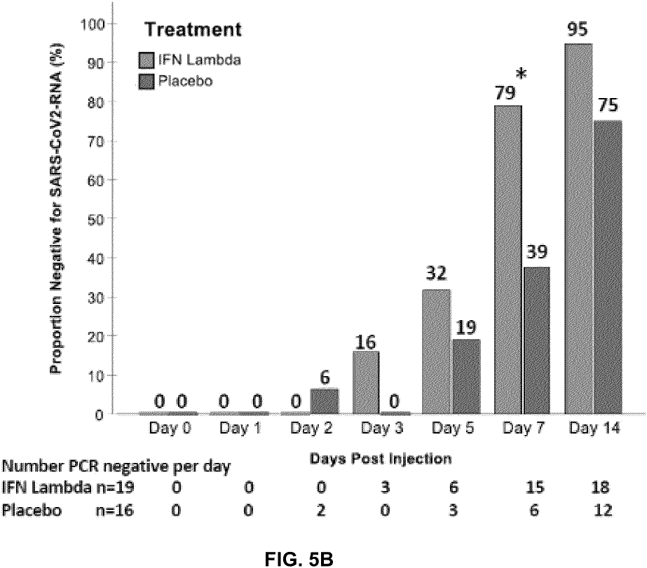

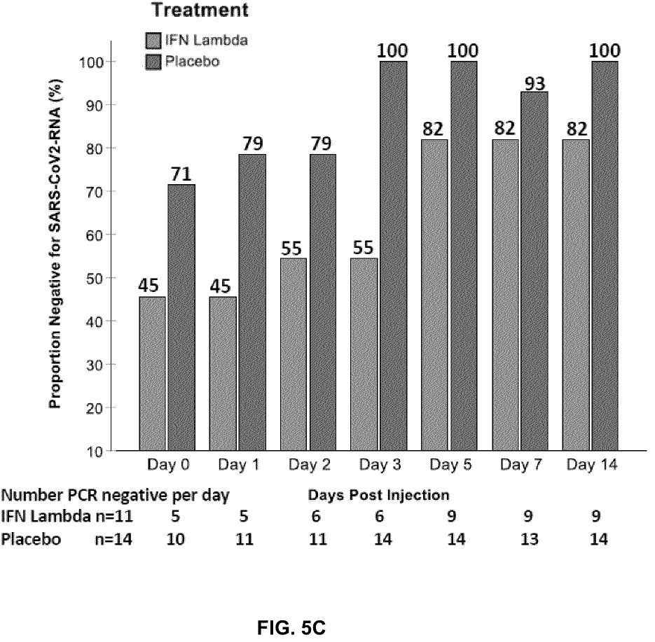

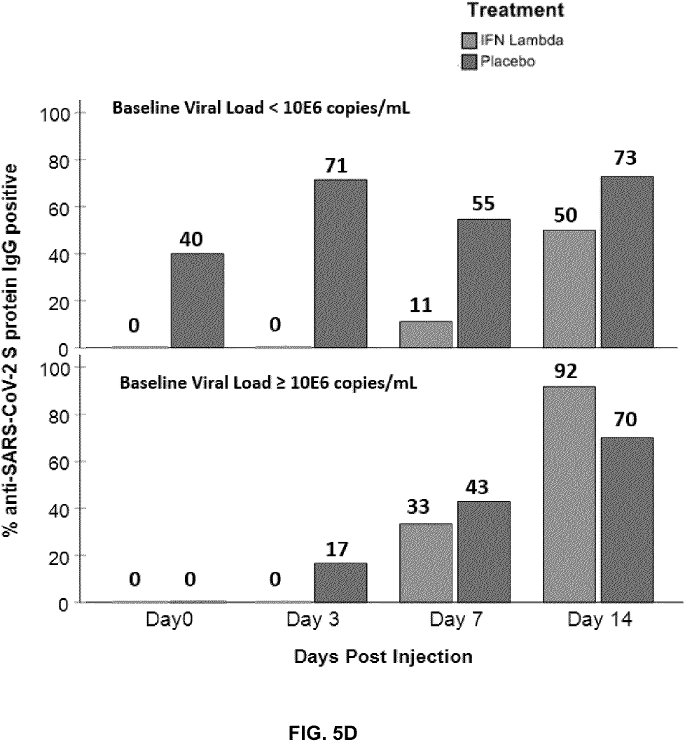

The decline in SARS-CoV-2 RNA was significantly greater in the peginterferon-lambda group than in the placebo group (p=0.04), as shown in FIGS. 3A-3F, with a similar effect observed when restricted to those with detectable virus at baseline as shown in FIGS. 3G-3H. The baseline SARS-CoV-2 RNA level, which was higher in the peginterferon lambda group, was associated with the probability of clearance by Day 7 (OR 0.69 95%CI 0.51-0.87, p=0.001). By Day 3, the viral load decline was 0.82 log copies/mL greater in the peginterferon-lambda group (p=0.14). By Day 5, the viral load decline was 1.67 log copies/mL greater in in the peginterferon-lambda group, and by Day 7 (p=0.013), the viral load decline was 2.42 log copies/mL greater in the peginterferon-lambda group (p=0.004). At Day 14, the difference in the viral load decline was 1.77 log copies/mL greater in the peginterferon-lambda group. In absolute terms, by Day 7 the viral level decreased by 5.5 log copies/mL in the peginterferon-lambda group, compared to 3.1 log copies/mL in the placebo group. At Day 14, the difference in viral decline was 1.77 log copies/mL greater in the peginterferon-lambda-treated group (p=0.048), as shown in FIG. 5B. The difference in viral load decline between groups was greatest in those with baseline viral load at or above 10E6 copies/mL, with a decline by Day 7 of 7.17 log copies/mL with peginterferon-lambda compared to 4.92 log copies/mL (p=0.004).

Overall, by Day 7, 24/30 (80%) in the peginterferon-lambda group were negative for SARS-CoV-2 RNA compared to 19/30 (63%) in the placebo arm (p=0.15), as shown in FIG. 5A. However, after adjusting for baseline viral load, peginterferon-lambda treatment was significantly associated with clearance by Day 7 (OR=4.12 95% CI 1.15-16.7, p=0.029), as summarized in Table 3.

TABLE 3

Adjusted effect of treatment on response at Day 7, overall, and in subgroup with viral load ≥10E6 copies/mL at baseline

| | All patients | | ≥10E6 copies/mL | |
|---|---|---|---|---|
| Model | OR 95% Cl | p-value | OR 95% Cl | p-value |
| Crude | | | | |
| Peginterferon Lambda vs Placebo | 2.32 (0.74, 7.81) | 0.150 | 6.25 (1.49, 31.06) | 0.012 |
| Adjusted for viral load at baseline | | | | |
| Peginterferon Lambda vs Placebo | 4.12 (1.15, 16.73) | 0.029 | 8.16 (1.76, 1.21) | 0.006 |
| Viral load at baseline | 0.69 (0.51, 0.87) | 0.001 | 0.60 (0.26, 0.00) | 0.157 |

Adjusted for viral load at baseline with interaction

| Peginterferon Lambda vs Placebo | 10E3 | 1.45 (0.12, 23.96) | 0.760 | | |
|---|---|---|---|---|---|
| By viral load (copies/mL) at baseline: | 10E4 | 1.88 (0.24, 18.81) | 0.531 | | |
| | 10E5 | 2.43 (0.47, 15.46 ) | 0.280 | | |
| | 10E6 | 3.14 (0.81, 14.03) | 0.096 | | |
| | 10E7 | 4.05 (1.15, 15.81) | 0.029 | | |
| | 10E8 | 5.23 (1.32, 23.52) | 0.018 | | |
| | 10E9 | 6.76 (1.29, 41.67) | 0.023 | | |
| Viral load at baseline; IFN Lambda | | 0.82 (0.50, 1.13) | 0.259 | | |
| Viral load at baseline; placebo | | 0.63 (0.40, 0.85) | 0.001 | | |
| Adjusted for sex | | | | | |
| Peginterferon Lambda vs Placebo | | 2.36 (0.75, 8.04) | 0.142 | 6.44 (1.50, 33.50) | 0.012 |
| Female vs Male | | 1.52 (0.47, 5.23) | 0.488 | 2.13 (0.47, 10.85) | 0.330 |

Adjusted for age

| | | | | |
|---|---|---|---|---|
| Peginterferon Lambda vs Placebo | 2.48 (0.78, 8.61) | 0.125 | 9.42 (1.91, 66.78) | 0.005 |
| Age (1 yr increase) | 0.97 (0.93, 1.02) | 0.226 | 0.95 (0.87, 1.02) | 0.151 |

Adjusted for IFNL4 genotype

| | | | | |
|---|---|---|---|---|
| Peginterferon Lambda vs Placebo | 2.24 (0.70, 7.72) | 0.175 | 7.66 (1.65, 45.65) | 0.008 |
| IFN4L_G & TT/ G vs TT | 0.78 (0.22, 2.54) | 0.677 | 0.46 (0.08, 2.24) | 0.340 |

Adjusted for BMI

| | | | | |
|---|---|---|---|---|
| Peginterferon Lambda vs Placebo | 2.37 (0.75, 8.06) | 0.142 | 6.73 (1.55, 35.90) | 0.010 |
| BMI <25 vs >=30 | 1.59 (0.31, 8.09) | 0.572 | 2.19 (0.23, 23.69) | 0.493 |
| BMI 25-29.9 vs >=30 | 1.22 (0.26, 5.31) | 0.792 | 1.37 (0.16, 11.55 ) | 0.767 |

Adjusted for comorbidity

| | | | | |
|---|---|---|---|---|
| Peginterferon Lambda vs Placebo | 2.35 (0.75, 7.98) | 0.144 | 10.46 (2.05, 82.63) | 0.004 |
| comorbidity no vs yes | 1.41 (0.26, 6.46) | 0.665 | 7.39 (0.78, 99.76 ) | 0.082 |

Adjusted for Asymptomatic at baseline

| | | | |
|---|---|---|---|
| Peginterferon Lambda vs Placebo | 2.91 (0.88, 10.79) | 0.081 | 0.00 (0.00, 0.00) |
| Asymptomatic at baseline: yes vs no | 2.13 (0.45, 15.64) | 0.357 | 0.00 (0.00, 0.00) |

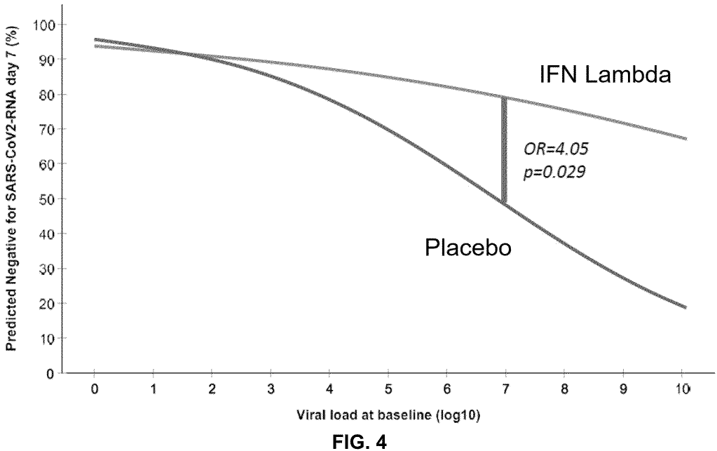

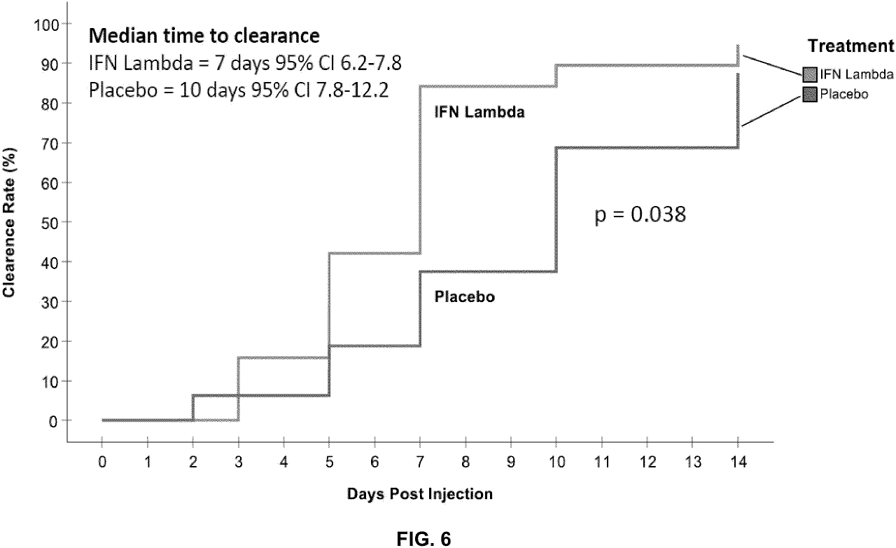

The odds of viral clearance by Day 7 with peginterferon-lambda treatment compared to placebo increased with every log increase in baseline viral load as shown in FIG. 4. For those with baseline RNA above 10⁶ copies/mL (58% of study population), the proportion undetectable at Day 7 in the peginterferon-lambda group was 15 of 19 (79%), compared to 6 of 16 (38%) in the placebo group (OR 6.25 95% Cl 1.49-31.1, p=0.012) as shown in FIG. 5B, translating to a median time to viral clearance of 7 (95% CI 6.2-7.8) days with peginterferon-lambda treatment compared to 10 (95% CI 7.8-12.2) days with placebo (p=0.038) as shown in FIG. 6. The mean log decline in SARS-CoV-2 RNA was greater with peginterferon-lambda than placebo from Day 3 onwards, with more pronounced differences seen in those with a high baseline viral load. The median time to clearance of SARS-CoV-2 RNA was 7 days in the peginterferon-lambda group compared to 10 days in the placebo group, amongst those with a high baseline viral load. In those with high baseline viral load, 3 of 4 participants in the peginterferon-lambda group with detectable virus at Day 7 had levels below 10⁴ copies/mL and 1 had 5.9E5 copies/mL. Of the 11 participants who remained positive at Day 7 in the placebo group, the viral load was above 10⁵ copies/mL in 6 and above 10⁶ copies/mL in 1 individual.

In contrast, in those with baseline viral load below 10⁶ copies/mL at baseline, 9 of 11 (82%) in the peginterferon-lambda arm and 13 of 14 (93%) in the placebo arm were undetectable at Day 7 (OR 0.35, 95%CI 0.01-4.15, p=0.40), as shown in FIG. 5C. Clearance was rapid in these subjects with no clear difference between those treated with peginterferon-lambda or placebo. It is notable that 25% of participants had undetectable viral loads by the time of study entry despite having a positive nasopharyngeal swab at the time of initial testing. Peginterferon-lambda was well-tolerated with a similar side effect profile to placebo. Treatment led to a higher rate of transient aminotransferase elevations, as has been previously reported, but was not associated with any other notable laboratory adverse events. There was a trend toward clinical improvement with peginterferon therapy with fewer emergency room visits (1 vs 4) and more rapid improvement in respiratory symptoms (p=0.06) compared to placebo.

No baseline covariates modified the association between baseline viral load and treatment assignment with clearance by Day 7, as summarized in Table 3. Participants who were asymptomatic were more likely to have baseline viral loads below 10⁶ copies/mL than those with symptoms (91% vs 27%, p<0.001). At randomization, 5/51 (9.7%) participants with available samples were seropositive for SARS-CoV-2 IgG antibodies, of whom 4 had undetectable SARS-CoV-2 RNA. Antibody positivity increased in both groups over time as shown in FIG. 5D. The presence of antibodies at any time point was associated with a corresponding lower viral load.

Participants with low viral loads also had milder symptoms at baseline with symptoms improving over time in both groups. Interferon lambda was well-tolerated with few adverse events, which included minimal elevations of transaminases which self-resolved.

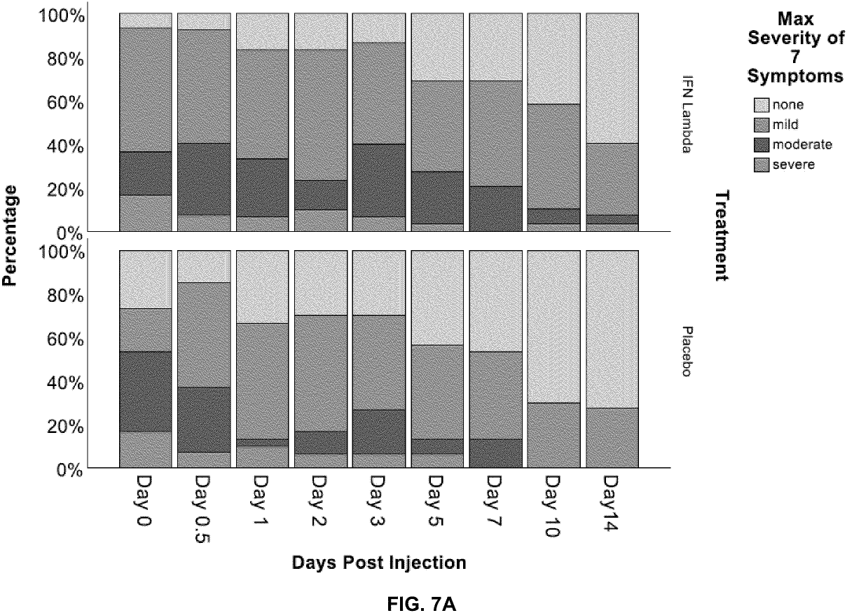

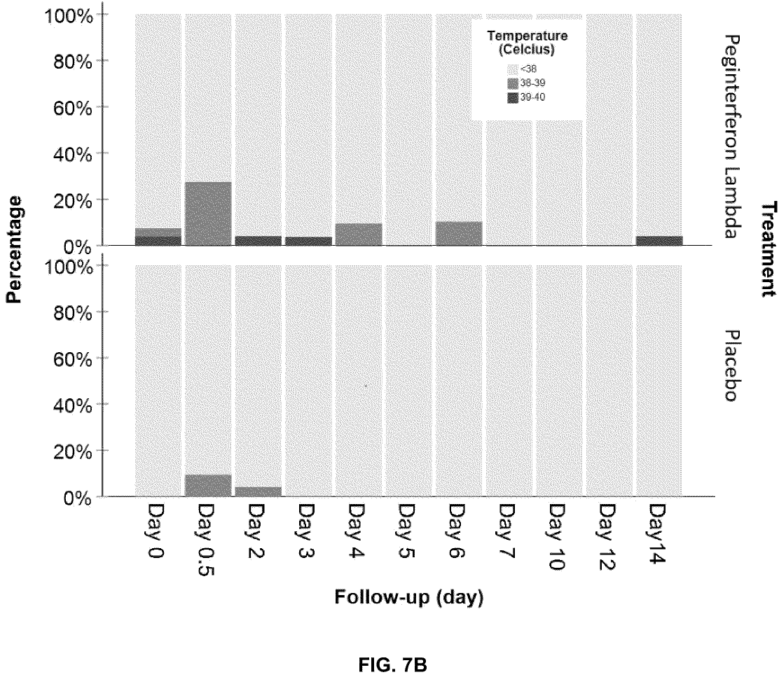

Symptoms were grouped into 7 categories and reported as absent/mild/moderate or severe, as shown in Table 4. Respiratory and fever-syndrome symptoms were most common in both groups, as shown in FIG. 7A. Documented temperature above 38° C. was rare but only reported beyond Day 2 in the peginterferon-lambda group as shown in FIG. 7B. Overall, most symptoms in both groups were mild and there was no difference in frequency or severity of any of the 7 symptom categories between treatment groups as summarized in Table 5. A symptom was graded as severe on 20 occasions by 7 patients in the peginterferon-lambda group and on 30 occasions by 7 patients in the placebo group. Symptoms improved in both groups over time, as shown in Table 5 and FIG. 7A. Participants with baseline viral loads above 10E6 copies/mL had higher symptom scores in all categories, except skin symptoms, than those with low baseline viral loads, as summarized in Table 5.

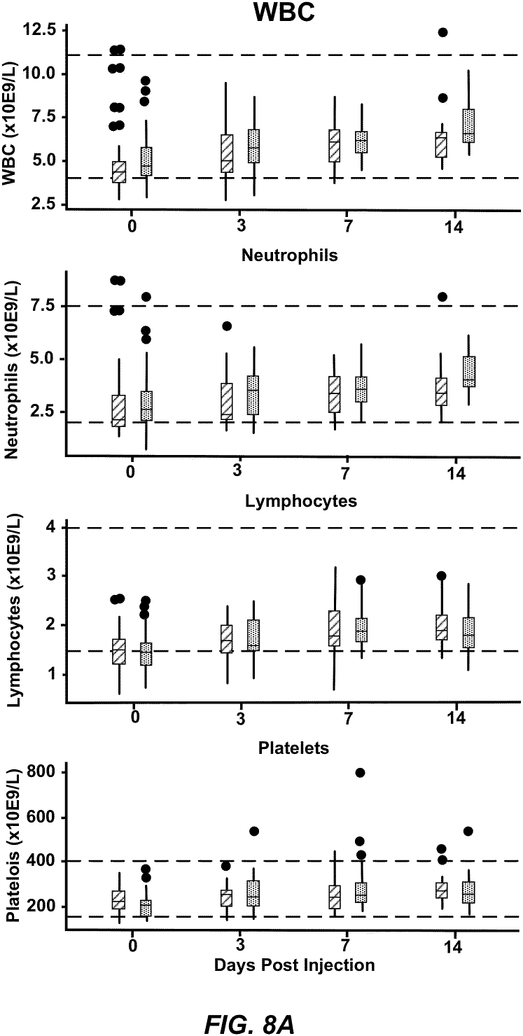

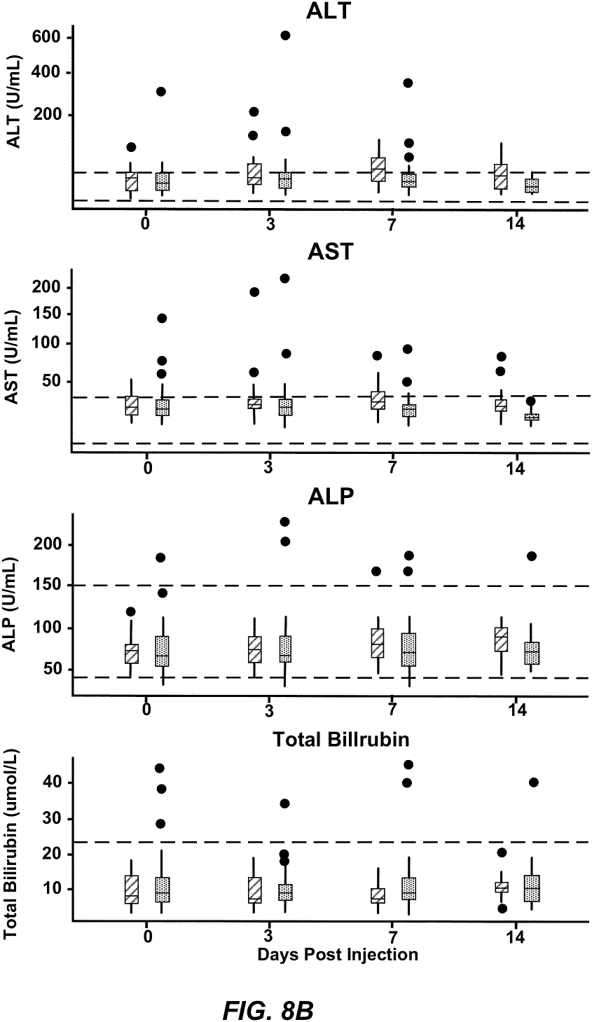

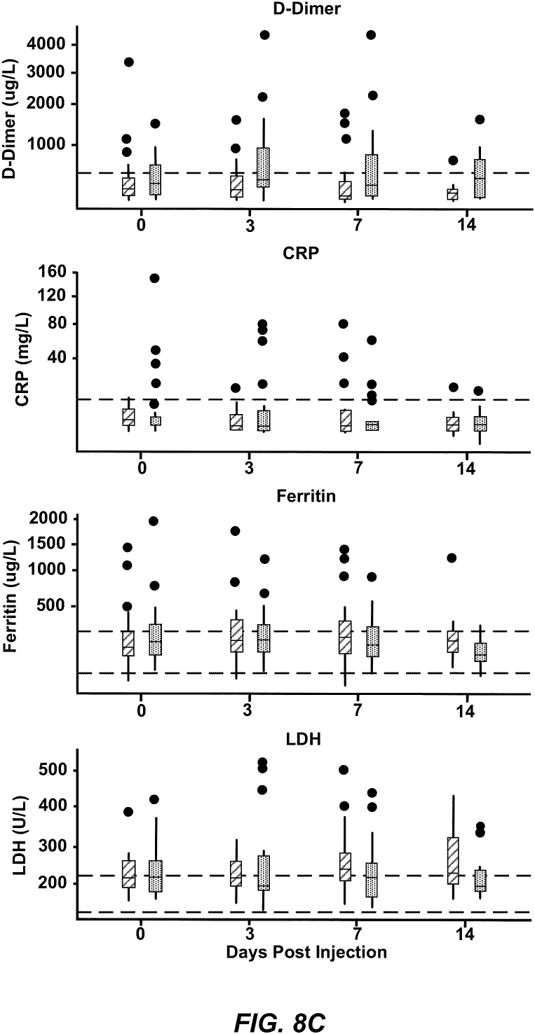

Laboratory AEs were mild and similar between groups. Aminotransferases were elevated at baseline in 3 (11%) participants in both groups and increased mildly, moreso in the peginterferon-lambda group, however only two individuals met the threshold of Grade 3 elevation, one in each arm. No other grade 3 or 4 laboratory AEs were reported, as summarized in Table 6. There were no elevations in direct or total bilirubin with the observed aminotransferase increases. Hemoglobin, white blood count and platelets were similar between groups with no episodes of myelosuppression in either group. D-dimers were elevated in both groups at baseline but declined over time only in the peginterferon-lambda group (Day 7: placebo 841 ug/L vs peginterferon-lambda 437 ug/L, p=0.02). Other inflammatory markers including ferritin and C-reactive protein were elevated at baseline in both groups and changed minimally over time, as shown in FIG. 8C.

AEs outside of the directed symptom categories occurred in one participant in the placebo arm (rectal bleeding) and in two in the peginterferon-lambda arm (confusion, pneumonia), all deemed unrelated to treatment. One serious adverse event was reported in each group. A participant in the placebo group was hospitalized on Day 1 post-injection with progressive shortness of breath attributed to worsening COVID-19. One participant in the peginterferon-lambda group was admitted to hospital on Day 14 with dyspnea and found to have a pulmonary embolism necessitating anticoagulation. No deaths occurred in either group.

TABLE 4

Categorization of daily symptoms that were assessed and whether they were likely due to COVID-19 or peginterferon lambda

| | | | |
|---|---|---|---|
| Symptom Category | Symptom | COVID-19 | Interferon |
| Fever Syndrome | Fever | X | X |
| | Chills | X | X |
| | Rigors | X | X |
| | Fatigue | X | X |
| Respiratory | Cough | X | |
| | Sore Throat | X | |
| | Shortness of breath | X | |
| | Chest pain | X | |
| | Runny nose | X | |
| | Conjunctivitis | X | |
| Gastrointestinal | Abdominal pain | X | |
| | Nausea | X | |
| | Vomiting | X | |
| | Diarrhea | X | |
| Musculoskeletal | Muscle Pain | X | X |
| Skin | Rash | | X |
| | Itch | | X |
| | Injection Site Reaction | | X |
| Mood | Depressed Mood | X | X |
| Neurologic | Loss of smell | X | |
| | Loss of taste | X | |
| | Change in color of fingers/toes | X | |

TABLE 5

Association between intervention and symptom progression by viral load

| Symptom Category | All samples | | Baseline Viral Load <10E6 copies/mL | | Baseline Viral Load ≥10E6 copies/mL | |
|---|---|---|---|---|---|---|
| | OR (95% CI) | p-value | OR (95% CI) | p-value | OR (95% CI) | p-value |
| All Symptoms | | | | | | |
| PeglFN-λ vs Placebo | 3.15 (0.77-12.96) | 0.11 | 1.63 (0.15-17.50) | 0.68 | 2.39 (0.48-11.82) | 0.28 |
| Days to improvement | 0.68 (0.60-0.76) | <0.0001 | 0.75 (0.61-0.92) | 0.0065 | 0.65 (0.55-0.78) | <0.0001 |

Fever Syndrome

| | | | | | | |
|---|---|---|---|---|---|---|
| PegIFN-λ vs Placebo | 1.69 (0.42-6.81) | 0.46 | 4.83 (0.11-204.88) | 0.41 | 1.24 (0.03-5.23) | 0.76 |
| Days to improvement | 0.67 (0.57-0.78) | <0.0001 | 0.59 (0.48-0.73) | <0.0001 | 0.76 (0.66-0.87) | 0.0003 |

Respiratory

| PegIFN-λ vs Placebo | 4.67 (0.91-23.91) | 0.06 | 3.67 (0.19-70.11) | 0.39 | 5.88 (0.81-42.46) | 0.079 |
|---|---|---|---|---|---|---|
| Days to improvement | 0.58 (0.48-0.71) | <0.0001 | 0.46 (0.23-0.94) | 0.035 | 0.68 (0.56-0.83) | 0.0004 |
| Gastrointestinal | | | | | | |
| PeglFN-λ vs Placebo | 0.48 (0.09-2.62) | 0.39 | 0.40 (0.0-123.14) | 0.75 | 0.58 (0.09-3.60) | 0.55 |
| Days to improvement | 0.72 (0.56-0.92) | 0.0090 | 0.62 (0.12-3.25) | 0.56 | 0.65 (0.48-0.88) | 0.0069 |
| Musculoskeletal | | | | | | |
| PegIFN-λ vs Placebo | 1.80 (0.45-7.26) | 0.41 | 2.55 (0.05-129.47) | 0.64 | 1.19 (0.35-4.08) | 0.78 |
| Days to improvement | 0.51 (0.35-0.76) | 0.0013 | 0.51 (0.18-1.46) | 0.20 | 0.49 (0.32-0.75) | 0.0016 |
| Skin | | | | | | |
| PegIFN-λ vs Placebo | 0.76 (0.10-5.92) | 0.80 | 0.51 (0.03-9.44) | 0.65 | 1.90 (0.08-48.08) | 0.69 |
| Days to improvement | 0.88 (0.59-1.33) | 0.5428 | 1.01 (0.89-1.16) | 0.87 | 0.94 (0.79-1.11) | 0.4387 |
| Neurologic | | | | | | |
| PegIFN-λ vs Placebo | 5.07 (0.24-108.01) | 0.30 | 1.60 (0.01-266.46) | 0.86 | 2.61 (0.07-92.85) | 0.59 |
| Days to improvement | 0.68 (0.61-0.75) | <0.0001 | 0.55 (0.41-0.74) | 0.0001 | 0.67 (0.58-0.77) | <0.0001 |

Mood

| | | | | | | |
|---|---|---|---|---|---|---|
| PegIFN-λ vs Placebo | 0.90 (0.10-8.35) | 0.93 | 0.32 (0.02-4.71) | 0.40 | 1.29 (0.07-25.62) | 0.87 |
| Days to improvement | 0.50 (0.21-1.18) | 0.1104 | 0.43 (0.21-0.89) | 0.0243 | 0.60 (0.30-1.17) | 0.1278 |

*The interaction between treatment group and days to symptom improvement were not statistically significant for any group or symptoms type.

TABLE 6

Summary of Adverse Events (AEs) and Severe Adverse Events (SAEs) by treatment group

| Type of Event | Intervention | |
|---|---|---|
| | Peginterferon Lambda | Placebo |
| Severe Symptoms | | |
| Reports (number of patients*) | 20(7) | 30(7) |
| AEs | 2 | 1 |
| SAEs | 1 | 1 |
| Treatment-Related AEs | 0 | 0 |
| Treatment-Related SAEs | 0 | 0 |
| Emergency Room visits | 1 | 4 |
| Hospital admissions | 1 | 1 |
| Lab Abnormalities/Toxicology (grade 3 or 4) | | |
| Hemoglobin | 0 | 0 |
| White Blood Cells | 0 | 0 |
| Lymphocytes | 0 | 0 |
| Neutrophils | 0 | 1 |
| Platelets | 0 | 0 |
| Creatinine | 0 | 0 |
| Alanine aminotransferase (ALT) | 1 | 3 |
| Aspartate aminotransferase (AST) | 1 | 1 |
| Total bilirubin | 0 | 0 |

*Total number of patients that reported severe symptoms throughout the study. Some patients reported multiple symptoms.

Treatment with a single dose of peginterferon-lambda accelerated the viral load decline and, after controlling for baseline viral load, reduced the time to viral clearance in outpatients with COVID-19. The treatment effect was most apparent in those with high baseline viral loads. Peginterferon-lambda was well tolerated with similar symptoms reported to those treated with placebo.

Results for SARS-CoV-2 diagnostic testing are routinely reported dichotomously as positive or negative without viral load quantification. The current standard of reporting cycle threshold (Ct) values is only semi-quantitative, and therefore assays or even runs cannot be reliably compared. Quantification is useful clinically as higher viral levels have been correlated with greater severity of COVID-19 and the level of virus correlates with infectivity. As people clear the virus, they may have persistently very low levels of RNA detected at very high Ct values (>33), which are not infectious.

Although the second trial found that, after controlling for baseline viral load, the odds of clearance were greater in all study participants with peginterferon-lambda than with placebo, the effect of peginterferon-lambda was most evident when baseline viral loads were above 10⁶ copies/mL. While the specific threshold for transmissible virus is unknown, using a standard infectivity assay, Bullard and colleagues reported that at Ct values above 24, corresponding to approximately 6-7 log copies/mL, infectious virus could not be detected. See Bullard et al., “Predicting infectious SARS-CoV-2 from diagnostic samples,” Clin. Infect. Dis., May 2020 (doi:10.1093/cid/ciaa638). It was observed that in individuals with low levels of virus, irrespective of their assigned group, spontaneous clearance occurred rapidly and near-universally by Day 7. Similarly, a recent evaluation of the REGN-COV2 monoclonal antibody cocktail demonstrated that individuals with the highest baseline viral loads exhibited the largest reduction in SARS-CoV-2 RNA with treatment, while those with detectable SARS-CoV-2 antibodies at baseline had low viral loads and did not benefit from therapy. See “Regeneron’s REGN-COV2 Antibody Cocktail Reduced Viral Levels and Improved Symptoms in Non-Hospitalized COVID-19 Patients,” Press Release, Regeneron Pharmaceuticals, Inc., Sept. 29, 2020, available at https://investor.regeneron.com/news-releases/news-release-details/regenerons-regn-cov2-antibody-cocktail-reduced-viral-levels-and.

In the placebo group with high baseline viral load, 10 of 16 (63%) participants had detectable virus at Day 7, with 6 of 10 (60%) continuing to exceed 10⁵ copies/mL, raising concern of persistent shedding of competent virus. In contrast, only 4 of 19 (21%) participants who received peginterferon-lambda had detectable virus at Day 7, all with viral loads below 10⁶ copies/mL. If this effect is confirmed in larger studies, a strategy of reserving treatment for those with high viral loads (≥10⁶ copies/mL) may shorten the required period of isolation with a reduced likelihood of transmission for all infected individuals. While quantitative testing could likely be introduced wherever quantitative PCR is used for diagnosis and may have an added benefit by predicting those at risk of a severe clinical course, it is currently not widely available. Given the tolerability of a single dose of peginterferon-lambda, it may be reasonable to consider treatment irrespective of baseline viral load, as a simple, universal approach. Alternatively, a qualitative assay, ideally a point-of-care test, could be titrated to achieve an analytical sensitivity of approximately 10⁶ copies/mL allowing for immediate risk stratification and determination of the need for treatment. Indeed, this could likely already be achieved using currently available rapid antigen tests, which demonstrate detection sensitivities in the range of 10-50,000 copies/mL, safely below the infectious threshold but avoiding those with extremely low viral loads who are unlikely to require any intervention.

Peginterferon-lambda was well tolerated with no safety concerns identified. Because the side effects of peginterferon-lambda may overlap with COVID-19 symptoms, it is difficult to distinguish whether AEs were related to treatment or persistent infectious symptoms. With detailed serial symptom assessment, it was found that symptoms improved in both treatment groups over time without obvious differences. Notably, among those who were asymptomatic at baseline, there was no difference in AEs between the treatment and placebo groups. Mild, reversible transaminase elevations were seen more frequently in the peginterferon-lambda group, which have been reported previously with this agent. Intriguingly, D-dimer levels fell with peginterferon-lambda treatment, which may be relevant given the association of high levels with more severe disease and increased all-cause mortality. The side effect profile and absence of hematological toxicity is in keeping with the better tolerability of Type III interferons compared to Type I interferons, such as IFN-alpha/IFN-beta. Treatment with interferon lambda may be particularly attractive given reports that impaired interferon production and the presence of autoantibodies to interferon alpha are associated with severe COVID-19. See Bastard et al., “Auto-antibodies against type IIFNs in patients with life-threatening COVID-19,” Science, September 2020 (doi:10.1126/science.abd4585); Zhang et al., “Inborn errors of type IIFN immunity in patients with life-threatening COVID-19,” Science, September 2020 (doi:10.1126/science.abd4570); Hadjadj et al., “Impaired type I interferon activity and inflammatory responses in severe COVID-19 patients,” Science, August 2020 (doi:10.1126/science.abc6027). Additional benefits include the broad activity of interferon lambda against multiple respiratory pathogens, including influenza, the very high barrier to resistance of interferon lambda, and the availability a long-acting formulation that permits a single subcutaneous injection.

Considerations for the study of the second trial: The sample size was small, but clearance rates in those with high viral loads were in line with the power calculations. Based on viral load and antibody data at the baseline visit, several participants were likely clearing or had cleared the virus, an observation reported in other COVID-19 outpatient studies. The benefit of treatment was mainly observed in the group with a high baseline viral load, requiring the introduction of quantitative assays or calibrated qualitative tests for COVID-19 diagnosis and risk stratification to operationalize its use. However, even those with low viral loads could be treated given the safety profile. A high proportion of potentially eligible individuals declined to participate in the study, likely based on the listed AE profile, which reflected weekly injections for a year for treatment of hepatitis B and C infections. Importantly, the enrolled population was diverse, with individuals born in 25 different countries.

### Example 7 Third Trial Results

A third trial will be performed using the methods and criteria described below.

TABLE 7

Third Trial Protocol

| | |
|---|---|
| OBJECTIVES | 1. To compare the proportion of patients with a COVID-19-related urgent care visit, emergency room assessment, hospitalization, treatment with an approved therapy (triggered by worsening COVID1-related signs and symptoms), or death by Day 28 in those treated with peginterferon lambda 180 mcg vs. those who receive placebo. |
| | 2. To compare the time to negativity for SARS-CoV-2 RNA post-treatment in those treated with peginterferon lambda 180 mcg vs. those who receive placebo. |
| STUDY DESIGN AND INTERVENTION | Individuals diagnosed with COVID-19 who are discharged home and symptomatic on the day of planned injection will be offered study enrollment. To enrich the population for people at risk of more severe outcomes, those with risk factors for severe COVID-19 will be recruited, including those with at least one of the following risk factors for severe COVID-19: age>55, hypertension/diabetes/obesity or severe symptoms at presentation (documented fever and/or respiratory symptoms including cough, shortness of breath and/or pleuritic chest pain and/or myalgias). Providers caring for patients with COVID-19 may also refer patients to the study team after obtaining verbal consent to share contact information with the study team. After speaking with research staff, eligible consenting participants confirmed to be COVID-19 positive by either a molecular test or point-of-care (POC) test on the Abbott ID NOW will be randomized. Those with a diagnosis prior to randomization will have a POC test performed prior to randomization. Randomization will be stratified by positive or negative POC test results with a maximum of 30% included with a negative POC test, as the correlation between viral load and ID NOW has not been fully characterized. As such symptomatic patients who are negative by this POC test may still benefit from treatment. |
| | Participants will be randomized to receive a single subcutaneous (SC) injection of peginterferon lambda 180 mcg or placebo. The peginterferon lambda and the saline will each be administered SC in the lower abdomen. Participants will complete daily symptom scores and will be followed through phone/videoconferencing. Self-collected mid-turbinate (MT) nasal swabs will be collected daily to Day 28. Virtual visits will occur on Days 1, 3, 5, 7, 10, 14, 17, 21, 24, 28. Participants will return to the ambulatory clinic on Day 7, 14, 28 and 60 for blood work and clinical evaluation. The primary clinical endpoint will be the proportion of participants with a COVID-19-related urgent care visit, emergency room assessment, hospitalization, or death by Day 28 and the primary virological endpoint will be the time to negativity in SARS CoV-2 RNA by MT swab. |
| NUMBER OF PATIENTS | n = 610 |
| STUDY DURATION | Approximately 3 months (2 months for enrollment, 4 weeks of follow-up). |
| ELIGIBILITY CRITERIA | Inclusion Criteria |
| | 1. Adult 18 years of age or older. |
| | 2. Symptomatic and within 5 days of symptom onset. |
| | 3. Confirmed COVID-19 infection by POC test or PCR within 5 days of enrollment with persistent symptoms at study entry. |
| | 4. High risk for severe disease (as defined by one or more of the following): |
| | a. Age >55 and/or |
| | b. HTN/DM/BMI>30 and/or |
| | c. Documented fever (>38° C.) and/or |
| | d. One of the following symptoms: cough, shortness of breath (SOB), pleuritic chest pain and/or myalgias |
| | 5. Discharged to home isolation. |
| | 6. Willing and able to provide informed consent (including by substitute decision maker). |
| | 7. Willing and able to follow-up by phone or videoconference. |
| | 8. Female patients of childbearing potential and male patients with partners of childbearing potential must agree to use adequate methods of contraception during the study and through 90 days after the last dose of study medication. Female patients of childbearing potential are all those except patients who are surgically sterile, who have medically documented ovarian failure, or who are at least 1 year postmenopausal. |
| | Exclusion Criteria |
| | 1. Current immunosuppression due to medication (steroids, biologics, chemotherapy) or underlying condition such as organ/bone marrow transplant or untreated HIV or HIV infection with detectable HIV RNA and/or CD4 count of <500. |
| | 2. Pregnancy (or positive urine pregnancy test) or lactating. |
| | 3. The following pre-existing medical conditions: |
| | a. Known cirrhosis with any history of decompensation (ascites, variceal bleeding, or hepatic encephalopathy) |
| | b. Known chronic kidney disease with estimated creatine clearance < 30 mL/minute or need for dialysis |
| | c. Uncontrolled severe psychiatric disorder - schizophrenia, bipolar disorder, depression with prior suicidality |
| | d. Any other underlying medical (cardiac, liver, renal, neurological, respiratory) or psychiatric condition that in the view of the investigator would preclude use of peginterferon lambda |
| | 4. Known alcohol or drug dependence that in the opinion of the investigator would impair study participation. |
| | 5. Known prior intolerance to interferon treatment. |
| | 6. Enrolment in another clinical trial testing an antiviral agent or receipt of an antiviral agent for COVID-19 in the past 30 days. |
| | 7. Use of off-label therapy for COVID-19. |
| PRIMARY ENDPOINTS | Primary efficacy endpoint: |
| | Proportion with COVID-19-related urgent care visit, emergency room assessment, hospitalization, or death by Day 28. |
| | Primary virological endpoint: |
| | The time to SARS-CoV-2 RNA negativity. |
| | Primary safety endpoint: |
| | The rate of treatment-emergent and treatment-related serious adverse events (SAEs) by Day 28. |
| SECONDARY ENDPOINTS | Secondary Endpoints: |
| | Clinical |
| | 1. Time to resolution of respiratory symptoms. |
| | 2. Time to resolution of fever. |
| | 3. Time to resolution of all symptoms (return to usual state of health). |
| | 4. Proportion of days with oxygen saturation below 93% on room air by Day 28. |
| | 5. Change in symptom scores from day 0 to Day 28. |
| | 6. Proportion seeking care from primary care provider/walk-in clinic or study healthcare provider for COVID-19 by Day 28. |
| | 7. Emergency room visit for COVID-19 from Day 0 to 28. |
| | 8. Duration of hospital admission up to Day 28. |
| | 9. Duration of intensive care admission up to Day 28. |
| | 10. Symptom score at Day 28. |
| | 11. Symptom score at Day 90. |
| | 12. Adverse events and serious adverse events by day 14. |
| | Virologic/Immunological |
| | 13. Time to SARS-CoV-2 RNA negativity. |
| | 14. Difference in mean SARS-CoV-2 RNA in log copies/mL by day 3, 5, 7, 10 and 14. |
| | 15. Difference in mean log decline in SARS-CoV-2 RNA by day 3, 5, 7, 10 and 14. |
| | 16. Proportion negative for SARS-CoV-2 RNA on day 3. |
| | 17. Proportion negative for SARS-CoV-2 RNA on day 7. |
| | 18. Proportion negative for SARS-CoV-2 RNA on day 14. |
| | 19. Proportion with SARS-CoV-2 antibodies in blood at day 0, 7, 14 and 90. |
| | 20. Correlation of response with interferon lambda 4 (IFNL4) genotype. |
| | 21. Change in laboratory and inflammatory markers (hemoglobin, white blood cell count, lymphocyte count, liver profile, ferritin, lactate dehydrogenase, c-reactive protein, D-dimers, creatine kinase) from day 0 to day 7 and day 7 to 14. |
| | Transmission |
| | 22. Confirmed diagnosis of COVID-19 in household contacts from day 0 - 28. |
| SAMPLE SIZE | The effect size is based on the results of a trial of bamlanivimab for the treatment of SARS-CoV-2 (BLAZE trial), which showed a reduction in hospitalization from 15% in the placebo group to 4% in the treatment group. Despite the more potent antiviral effect of peginterferon-lambda, it was conservatively assumed a lower effect size of 50%. To achieve 80% power with alpha at 0.05, 277 patients per arm would be required to show a reduction from 15% to 7.5%. Assuming 10% dropout, the study would require 610 patients in total. Assuming a similar virological effect as seen in the original Phase 2 trial with peginterferon-lambda, with over 100 patients per arm with viral loads above 10E6, there will be >99% power to detect a difference in time to viral clearance. |
| RANDOMIZATION AND TREATMENT ASSIGNMENT | Participants will be randomly assigned 1:1 in blocks of 4 using centralized computer-generated randomization to receive either treatment with peginterferon lambda or placebo. Randomization will be stratified by result of POC COVID-19 test at enrolment (positive vs negative). |
| DATA TO CAPTURE | Demographic data: Age, sex, ethnicity, country of birth |
| | Living arrangements: house/apartment, long-term care, number of household contacts (adults and children), number of bedrooms |
| | Medical history – any pre-existing medical conditions with specific focus on history of diabetes, heart disease, lung disease |
| | Current prescribed medications and any drug allergies. |
| | Habits: smoking (none/current/past), recreational drug and alcohol use (none/current/past) |
| | Physical examination: Vital signs (pulse, blood pressure, respiratory rate, oral temperature, oxygen saturation), weight & height |
| | Routine blood tests: complete blood count, biochemistry (electrolytes, creatinine, liver enzymes, total and direct bilirubin and albumin) and inflammatory markers including D-Dimers, LDH, ferritin, c-reactive protein and creatine kinase. |
| DATA MANAGEMENT AND ANALYSIS | All analyses will be conducted with a modified intention-to-treat approach. Statistical inference will use a two-sided Type 1 error rate of 0.05 and 95% confidence intervals. |
| | The primary endpoint is the proportion of patients requiring ER visit or hospitalization for COVID-related signs and symptoms, or death on or before Day 28. |
| | Time to SARS-CoV-2 negativity will be determined by Kaplan-Meier analysis. |
| | Analysis of adverse event (AE) data will primarily be descriptive based on Medical Dictionary for Regulatory Affairs (MedDRA) coding of events. |

### 1. Consent Process

Where feasible, individuals at the COVID-19 assessment centers or emergency departments will be offered evaluation using the ID NOW POC COVID-19 test and provided with information about the study. Those testing positive by ID NOW will be offered immediate evaluation for eligibility and enrolment. Where POC testing in the assessment centre or emergency room is not available, individuals will be given written information about the study and provided information to contact study staff by email or telephone if they are interested in study participation. The study will also be advertised on social media with contact information of the study team. Providers caring for patients with COVID-19 may also refer patients to the study after obtaining verbal consent to share contact information with the study team. Those with a positive POC test or referred to the study with confirmed positive results will be screened by study staff for other inclusion/exclusion criteria and will be provided a consent form in person or electronically for review. In addition to the consent to the trial, participants will be offered an additional optional consent for genetic testing (see, infra, Ex. 7, Section 9) and a second optional consent for collection of peripheral blood mononuclear cells (“PBMCs”) at participating sites (see, infra, Ex. 7, Section 10). The study coordinator will read the consent form verbatim while the patient reads along. When the patient consents, the study coordinator will sign his/her copy of the document, and this will be witnessed and signed by an impartial witness. This document will stay in the patient’s study file. Upon coming to the clinic, the study team will provide the patient a paper copy to keep and not return to the study team. Those with a positive test for SARS-CoV-2 and who meet all inclusion/exclusion criteria and have signed consent will be randomized (see, infra, Ex. 7, Section 7).

### 2. Enrollment and Randomization

Those who test positive and remain symptomatic (to be called morning of visit for confirmation of persistent symptoms if no POC test is available in the assessment centre) will be invited to attend the outpatient clinic for completion of screening, enrolment and randomization (see, infra, Ex. 7, Section 7). Potential participants will be screened by phone for factors associated with severe COVD-19 (age>55 years, diabetes/hypertension/obesity, severe symptoms including documented fever and/or respiratory symptoms and/or myalgias). Consenting individuals will undergo a medical history evaluation, including current medication use, and complete a symptom survey to be recorded on a baseline case report form Women of childbearing potential will take a urine pregnancy test to confirm eligibility. Female participants who are concerned they may be pregnant will not be enrolled even if the test is negative (in case it is too early for a positive result). Female and male subjects will be advised to use appropriate measures to avoid pregnancy during the week following administration of peginterferon lambda and for at least 3 months after the dose of peginterferon lambda.

Vital signs, including blood pressure, temperature, pulse, respiratory rate and oxygen saturation in ambient air will be recorded. The eligibility checklist will be reviewed by a site sub-investigator (″sub-I)/principal investigator (“PI”) and if deemed to be necessary, a history and physical examination will be performed by the sub-I/PI. Potential participants meeting all inclusion and no exclusion criteria will be offered study enrolment. Eligible participants will have a POC COVID-19 test performed, a provider-collected NP swab for viral load quantification and will have blood drawn for routine laboratory (CBC, creatinine, liver profile) and inflammatory markers (LDH, ferritin, D-Dimers, c-reactive protein, creatine kinase), a research sample for plasma to be stored for future use, as well as optional blood for genetic and PBMC sub-studies (at participating sites) for those who consent. The genetic and/or PBMC sample will replace the research plasma sample, as plasma can be used after PBMC isolation or preparation for extraction of genetic material. Patients will also be instructed on self-collection of mid-turbinate nasal swab and will self-swab witnessed by the study staff.

Eligible patients included in the study will be assigned to one of the 2 treatment arms according to a standard computer-generated randomization schedule 1:1 in blocks of 4, stratified by POC test result (positive or negative). Numbered opaque envelopes with treatment arm allocation for randomized subjects will be stored at the outpatient site. Upon instruction to randomize from the PI or designate sub-I, the coordinator will open the envelope to reveal the treatment allocation. The study ID, month and year of birth and initials will be recorded on the randomization form as a unique identifier and emailed/faxed to the TCLD. The treatment codes will be maintained by the trial statistician in a password-protected file which cannot be accessed by other study personnel or subjects. In future study materials and analyses, the subject will be referred to only by the study identification number.

### 3. Study Interventions

Subjects randomized to the peginterferon lambda arm will receive a single SC injection in the lower abdomen of peginterferon lambda 180 mcg, and subjects randomized to placebo will receive a single SC injection in the lower abdomen of saline (and this will count as Day 0 of the study for the Schedule of Events shown in FIG. 9). After the injection, participants will be observed for 10 minutes to ensure there are no immediate complications from the medication. After 10 minutes of observation, participants will be discharged. Participants will be provided with additional swabs for mid-turbinate nasal swabs to be collected by self-collection as well as a sealable cooler for home swab storage until collection. The coordinator will confirm contact information for the participant with the number of an emergency contact as well, and the participants will be provided with the contact information to reach the study team. A detailed schedule for follow-up and a symptom assessments will be provided to the participant. If participants do not own a digital thermometer or oximeter, they will be given one by the study team.

After discharge, participants will follow the standard-of-care advice given to all individuals with COVID-19 at the Assessment Centre/ER. Participants will return home and remain in home isolation for at minimum 10 days from symptom onset according to current local Public Health recommendations. The exception to home isolation will be for study visits, as permitted by Public Health (for example, people with COVID-19 may attend medical appointments provided proper precautions are taken including wearing a mask at all times).

Participants will be contacted at a pre-specified time on multiple check days (proposed: days 1, 3, 5, 7, 10, 14, 17, 21, 24, and 28) by a study coordinator by phone/videoconference to review the symptom questionnaire and AE survey, concomitant medications, and to record the digital oral temperature and oxygen saturation. During the virtual visit, results will be recorded onto case report forms by the study coordinator and entered into the secure REDCap electronic case report form (eCRF) database. Participants will collect a mid-turbinate nasal swab after speaking to the study coordinator and, wherever possible, collection will be observed by the study coordinator using videoconferencing. On subsequent days, mid-turbinate nasal swabs will be self-collected without observation unless requested by the participant. The viral media with the swab will be stored in a plastic container inside a cooler that will be provided to the participant and stored until collection.

On Days 1-6, and 8-13, a self-collected mid-turbinate nasal swab will be taken and stored as above.

On Day 3, the self-collected mid-turbinate nasal swabs from Days 1, 2 and 3 will be retrieved by courier.

On Day 7, the participant will attend the outpatient clinic and a provider-collected NP swab and self-collected mid-turbinate nasal swab will be obtained. The Day 4, 5, and 6 mid-turbinate swabs will be retrieved at this visit. Blood will be drawn for routine laboratory and inflammatory markers, a research sample to be stored for future use and additional samples for those who consented to PBMC collection.

On Day 10, the self-collected mid-turbinate nasal swabs from Days 8, 9, and 10 will be retrieved by courier.

On Day 14 and/or Day 28, the participant will return to the outpatient clinic and a provider-collected nasopharyngeal swab and a self-collected mid-turbinate nasal swab will be obtained. The Day 11, 12, and 13 swabs will be retrieved at this visit. Blood will be drawn for routine laboratory and inflammatory markers, a research sample to be stored for future use and additional samples for those who consented to PBMC collection.

On Day 90+ (up to one year), participants will return to the outpatient clinic for a provider-collected NP swab, a final blood draw and to complete the symptom survey. Those who consent to Day 90 PBMC collection (even if they did not consent to PBMC collection during the treatment phase of the study) will have these additional tubes drawn. Up to 8 tubes of blood will be collected.

For participants who cannot travel to the clinic, the option of home visits by the study team will be discussed for the Day 7, 14, and/or 28 visits (see, infra, Ex. 7, Section 5). Home visits will not be offered for the Day 90 visit.

### 4. Mid-Turbinate Swab Collection

The rationale for self-collection of mid-turbinate nasal swabs is to allow for more frequent sampling to determine the time of viral clearance and quantitative viral kinetics. Self-collection of mid-turbinate nasal swabs has been validated and was performed in the prior in studies of peginterferon-lambda for COVID-19. Understanding how quickly individuals clear SARS-CoV-2 is very important for determining when people could potentially end their self-isolation. In addition, viral kinetics using quantitative PCR may provide insights into the mechanisms of clearance, as has been successfully done with other viral infections. A previous study done by our group has shown that mid-turbinate nasal swabs can be reliably self-collected with only marginally lower sensitivity (69/71, 91% concordance) for influenza, rhinovirus and respiratory syncytial virus detection than standard nasopharyngeal swab.

Participants will be instructed and observed at the first visit on the collection of mid-turbinate nasal swabs and if possible, the first self-collection sample will be observed by the coordinator during the videoconferencing visit. Participants will be given written instructions on how to store the swabs. After putting the swab into the viral culture media, the swab will be placed into two clear biohazard bags inside the provided sealed cooler. A courier will retrieve the Day 1, 2 and 3 swabs on Day 3 and similarly on Day 10, the Days 8, 9 and 10 samples will be retrieved. At the Day 7 and 14 clinic visits, the participant will bring in samples 4, 5 and 6 (for Day 7) and 11, 12 and 13 (Day 14). To do so, they will place the cooler into 2 provided clear biohazard bags and bring the bags to the clinic. Upon receipt of the bags in the clinic, the outer bag will be decontaminated and then the specimens will be taken or sent to Toronto General Hospital for storage at -80° C. in the laboratory of the PI.

### 5. Home Visits

For participants unable to travel to clinic visits, home visits by study staff will be provided as an alternative, provided participants live within a 30-minute drive of the site from which they were recruited and are agreeable to having study staff visit their home. Procedures/precautions will be taken to ensure staff safety. For home visits, the study coordinator will drive to the participant home at an agreed up pre-specified time. Upon arrival, the coordinator will call the participant as notification. The coordinator will don personal protective equipment (mask, gown, gloves and face shield) and enter the home to carry out the study visit. Upon completion of the study visit, the coordinator will doff personal protective equipment and place it in a clear plastic bag. It will then be transported back to the hospital/clinic for appropriate disposal.

### 6. Household Contacts

For participants with household contacts, the coordinator will ask the participant at each contact to report a confirmed diagnosis of COVID-19 with the date of symptom onset in any household contacts. Participants will also record diagnosis of COVID-19 in household contacts in their symptom diaries. Participants will be contacted at Day 28 to specifically ask if any household contacts have been diagnosed with COVID-19 and the date of symptom onset. For the purpose of analysis, any confirmed COVID-19 diagnoses in household contacts within 3 days of study enrolment will be considered to be present prior to the study and will not count in assessment of incident infections.

### 7. Clinic Visits

Safety procedures will be followed to ensure that clinic visits are carried out safely minimizing exposure to the public and study staff. Upon arrival, the participant will call the study coordinator from the car. The coordinator will advise the participant when to come into the clinic. For participants who are unable to drive to clinic visits, a chauffeur will be arranged by the study team.

### 8. Safety Assessment

Blood work will be collected prior to the peginterferon lambda injection but will not be used to determine eligibility. Hepatotoxicity has been noted in studies of peginterferon lambda in patients with chronic viral hepatitis. Transaminase elevations were reported, and hepatic decompensation has been reported but only in people with a prior history of decompensation prior to dosing. The study team has extensive experience managing patients with underlying liver disease (3 investigators are hepatologists) and multiple investigators have experience with peginterferon lambda use as well. If baseline laboratory results are suggestive of cirrhosis (unlikely), patients will be informed of this and followed carefully for signs of hepatic decompensation (ascites, hepatic encephalopathy, variceal hemorrhage) during follow-up, with prompt referral to the hospital should these signs/symptoms occur.

The most relevant other concern would be unrecognized renal impairment. Dosing advice is unclear for patients with estimated glomerular filtration rate (eGFR) below 50 mL/min. Participants found to have a reduced eGFR (<50 mL/min) after dosing will be advised of the test result and the need for follow-up. The consequences of dosing during renal impairment are not well understood but may lead to increased concentrations of systemic interferon lambda. Participants will be followed virtually frequently with in-person visits at day 7 and 14 with repeat blood tests on those days. Those with unexpected renal impairment will be followed according to the standard follow-up in the protocol, however, additional investigations may be performed at the discretion of the treating physician.

### 9. Optional Consent: Genetic Testing

A genome-wide association study (GWAS) performed on people treated with interferon-alpha therapy for hepatitis C virus (HCV) infection identified a single nucleotide polymorphism (SNP) near the interleukin 28B (IL28B) gene that was strongly associated with response to treatment. See Ge D et al., “Genetic variation in IL28B predicts hepatitis C treatment-induced viral clearance,” Nature, September 2009; 461(7262):399-401. doi:10.1038/nature08309. Subsequent studies confirmed the association and found that this SNP was also associated with spontaneous HCV clearance. See Thomas DL et al., “Genetic variation in IL28B and spontaneous clearance of hepatitis C virus,” Nature, October 2009; 461(7265):798-801. doi:10.1038/nature08463. Although the originally identified SNP was in a non-coding region, a later study identified a novel mRNA transcript induced by viral infection in hepatocytes. The transcript codes for a novel Type III interferon called interferon lambda 4 (IFNL4). See Prokunina-Olsson L et al., “A variant upstream of IFNL3 (IL28B) creating a new interferon gene IFNL4 is associated with impaired clearance of hepatitis C virus,” Nat. Genet., February 2013; 45(2):164-71. doi:10.1038/ng.2521. A deletion in the IFNL4 gene prevents production of a functional protein. The lack of the functional IFNL4 is associated with HCV treatment response to interferon-based therapy and with spontaneous HCV clearance. In contrast, production of functional IFNL4 is associated with non-response to interferon-based therapy for HCV. The prevalence of the IFNL4 mutation varies by ethnicity, with 80% of East Asians producing no functional IFNL4 whereas approximately 75% of Africans produce the functional protein. The difference in prevalence explains the bulk of the difference in HCV treatment response by ethnicity. It is unknown whether the IFNL4 genotype affects response to interferon lambda treatment and/or the natural course of COVID-19. Currently no other genes have been identified that modify the course or response to treatment of COVID-19.

Study participants will be asked to sign an optional consent giving permission to study genetic associations between disease outcome and treatment response during COVID-19 infection. Participants who agree to genetic testing will have a tube of whole blood taken on Day 0 for DNA extraction and storage. The IFNL4 genotype will be determined in all consenting participants and DNA will be stored for future analysis in case other relevant genes are identified.

### 10. Optional Consent: Peripheral Blood Mononuclear Cell

To evaluate SARS-CoV-2-specific immune responses, a subset (~30%) of participants will be asked to consent to provide additional blood for peripheral blood mononuclear cell (PBMC) isolation. Those who agree will have 5 ACD (acid citrate dextrose) tubes collected on Day 0, 7 and 14. The magnitude and change in SARS-CoV-2-specific immune responses will be evaluated using standard interferon-gamma ELISPOT assays to over-lapping peptides of SARS-CoV-2. Participants will be asked to consent to provide additional blood for PBMC isolation at Day 90+ post-dosing (up to 1-year post-dosing). Provision of blood for the Day 90+ PBMCs will be requested of all participants irrespective of whether they agreed to PBMC collection during the course of treatment. The rationale for the late PBMC collection is to assess the degree of T cell immunity and antibodies targeting SARS-CoV2 and to determine whether the PBMC responses are influenced by peginterferon lambda treatment.

### 11. Optional Consents: Antibody Testing

The presence of antibodies to SARS-CoV-2 will be assessed at Days 0, 7, 14, 28 and 90+. Although the clinical significance of the presence of IgM/IgG antibodies is not fully understood, the presence and quantity of anti-COVID-19 antibodies on day 14 of the study to day 90+ visits will be compared; including assessing whether the administration of peginterferon lambda may affect the emergence or quantity of antibody. In addition to collecting plasma, the utility of collecting blood by finger-prick onto a dried blood spot card will be assessed. The sample would be eluted from the card and will also be analyzed on one of the Health Canada approved platforms. Dried blood spots have been widely used in resource-limited countries for the presence of hepatitis B, C and HIV antibodies, and several countries are also implementing this collection method for seroprevalence studies of COVID-19. However, to date, there are few head-to-head comparisons of venipuncture to finger-prick collection for COVID-19 antibodies; and the ability to collect by both methods in this study will provide data as to whether this method is feasible and comparable to testing from plasma.

### 12. Study Withdrawal

An investigator may advise a participant to withdraw from the study if there are concerns for participant safety. Data from participants who discontinue for safety will still be collected unless the participant withdraws consent. Participants who discontinue prematurely before assessment of the primary endpoint will be counted as treatment failures for analysis. Participants who discontinue prematurely due to safety concerns will not be replaced.

Participants may withdraw from the study at any time. The reason for withdrawal must be documented. Participants who discontinue prematurely will be included in the analysis of results (as appropriate) and may be replaced in the enrollment. If agreeable, participants who choose to discontinue the study prematurely, will be asked to have a final study visit to document final virologic results. Participants may decline the final study visit at the time of withdrawal.

### 13. Data Analysis

The assessment of endpoints–safety, clinical and virological efficacy–will be determined by study staff blinded to the treatment assignment of the participant. Descriptive statistics will be used to summarize demographic and clinical baseline characteristics of enrolled participants. Continuous variables will be summarized with mean, median, SD, quartiles, and minimum and maximum values, as appropriate. Categorical variables will be summarized using counts and proportions. For the primary clinical endpoint, the association of peginterferon-lambda with ER/hospitalization will be evaluated by logistic regression as univariate analysis and as bivariate analysis controlling for baseline viral load. The primary virological outcome will be assessed with a log rank test comparing the two survival curves of SARS-CoV-2 RNA negativity over the first 14 days. Once Day 14 information has been collected for the last participant, the study will be unblinded and the data will be made available for analysis to allow for prompt dissemination of the results. Day 28 information will still be collected thereafter for the remaining participants, but this only pertains to outcomes of potential home transmission so will not influence the primary or key secondary outcomes. RNA negativity for determination of the primary virological endpoint will require two consecutive negative specimens but will be counted as occurring on the first of the two negatives. Participants who die before reaching RNA negativity will be counted as never reaching negativity. Participants who withdraw from the study prior to reaching RNA negativity will be counted as never reaching negativity for the ITT analysis. For the secondary endpoint of incident infection in household contacts, infections with symptom onset within 3 days of study enrollment will be deemed to have occurred prior to study enrollment and thus not counted as post-study enrolment incident infections.

A secondary analysis will be performed on the modified ITT population, including anyone who took a dose of peginterferon lambda or placebo. Factors associated with severity of disease and clinical course will be evaluated by uni- and multivariable logistic regression. Secondary endpoints will be described and analysed depending on the outcome with chi-2 test for proportions, log-rank test for time to event and repeated measurement modelling for multiple outcomes per patient over time. Viral kinetics will be determined using quantitative SARS-CoV-2 RNA and correlated with inflammatory and cytokine profiles. If feasible, quantitative results will be plotted to develop a model of peginterferon lambda activity against SARS-CoV-2. A complete statistical analysis plan will be created prior to data analysis.

### 14. Symptoms and AE/SAE Reporting

Symptoms will be collected by phone/videoconference or by self (depending on the study day). Participants will be asked about specific symptoms known to be common in COVID-19 or to be reported with interferon use. Symptoms will be rated as: none, mild, moderate or severe. They will also be asked about their overall state of health and an open-ended question about additional symptoms and again rate them by severity and change over time. The following symptoms will be specifically explored:
Shortness of breath
Cough
Chest pain
Fever
Chills
Stuffy/runny nose
Loss of smell
Loss of taste
Fatigue/weakness
Headache
Nausea
Loss of appetite
Vomiting
Diarrhea
Muscle pain/aches
Injection site reaction

An adverse event (AE) is any adverse change from the participant’s baseline (pretreatment) condition, including intercurrent illness which occurs during the course of the trial, after the consent form has been signed, whether the event is considered related to treatment or not. The Common Terminology Criteria for Adverse Events CTCAE v 5.0 will be used for grading severity of AEs.

A serious adverse event (SAE) is any adverse event that at any dose:
results in death (grade 5 event)
is life-threatening (grade 4 event)
requires inpatient hospitalization or prolongation of existing hospitalization
results in persistent or significant disability or incapacity
is a congenital anomaly/birth defect

Unexpected adverse events are those which are not consistent in either nature or severity with information contained in the investigator brochure or product monograph.

Adverse events considered related to protocol treatment are those for which a relationship to the protocol agent cannot reasonably be ruled out.

All serious adverse events which are unexpected and related to protocol treatment must be considered reportable, and therefore be reported in an expedited manner.

Medical and scientific judgment will be exercised in deciding whether expedited reporting is appropriate in other situations such as important medical events that may not be immediately life threatening or result in death or hospitalization but may jeopardize the patient or may require intervention to prevent one of the events listed above. These should also be considered serious.

All SAEs meeting the above criteria must be reported to the sponsor site to either the PI or the TCLD research coordinator in an expedited fashion. SAEs should be reported to the sponsor within 24 hours. In many instances, complete clinical information may not be available. Whatever information is available on the SAE should be provided to the sponsor within 24 hours. As new information becomes available, it should be forwarded to the sponsor. Each site will report unexpected AEs or SAEs to their REB as per their local site regulations.

A serious adverse event, which is unexpected and is related, will require expedited reporting to the appropriate oversight committees or entities, as per local site regulations.

In conclusion, this is the first antiviral therapy to show benefit among outpatients with COVID-19. Peginterferon-lambda accelerated viral clearance, particularly in those with a high viral load at baseline. This treatment has the potential to avert clinical deterioration, shorten the duration of infectiousness and limit time required in isolation.

According to various embodiments of this disclosure, additional information relating to diagnostic criteria, viral load, medical history, and relevance to disease severity can be found in:
Markus Mordstein et al, Lambda Interferon Renders Epithelial Cells of the Respiratory and Gastrointestinal Tracts Resistant to Viral Infections, JOURNAL OF VIROLOGY, June 2010, p. 5670-5677 Vol. 84, No. 11.
Scott A. Read, et al., Macrophage Coordination of the Interferon Lambda Immune Response, Frontiers in Immunology, November 2019, Volume 10, Article 2674.
Tanel Mahlako, et al., Combined action of type I and type III interferon restricts initial replication of severe acute respiratory syndrome coronavirus in the lung but fails to inhibit systemic virus spread, Journal of General Virology (2012), 93, 2601-2605.
Helen M. Lazear, et al., Interferon-I: Immune Functions at Barrier Surfaces and Beyond, Immunity 43, Jul. 21, 2015.
Ole J Hamming, et al., Interferon lambda 4 signals via the IFNk receptor to regulate antiviral activity against HCV and coronaviruses, The EMBO Journal (2013) 32, 3055-3065.
Paules C, Marston H, Fauci A. Coronavirus Infections - More Than Just the Common Cold, JAMA. Published online Jan. 23, 2020. doi:10.1001/jama.2020.0757.
Wang C, Horby P, Hayden F, Gao G. A novel coronavirus outbreak of global health concern, The Lancet, Published online Jan. 24, 2020. https://doi.org/10.1016/ S0140-6736(20)30185-9.
Chan JF, Yuan S, Kok K, et al. A familial cluster of pneumonia associated with the 2019 novel coronavirus indicating person-to-person transmission: a study of a family cluster, The Lancet. Published online Jan. 24, 2020. https://doi.org/10.1016/ S0140-6736(20)30154-9.
Huang C, Wang Y, Li X, et al. Clinical features of patients infected with 2019 novel coronavirus in Wuhan, China, The Lancet. Published online Jan. 24, 2020. https://doi.org/10.1016/ S0140-6736(20)30183-5.
Heymann D. Data sharing and outbreaks: best practice exemplified, The Lancet. Published online Jan. 24, 2020. https://doi.org/10.1016/ S0140-6736(20)30184-7.
The Lancet. Emerging understandings of 2019-nCoV, The Lancet. Published online Jan. 24, 2020. https://doi.org/10.1016/ S0140-6736(20)30186-0.
Chen N, Zhou M, Dong X, et al. Epidemiological and clinical characteristics of 99 cases of 2019 novel coronavirus pneumonia in Wuhan, China: a descriptive study, The Lancet. Published online Jan. 29, 2020. https://doi.org/10.1016/ S0140-6736(20)30211-7.
Lu R, Zhao X, Li J, et al. Genomic characterization and epidemiology of 2019 novel coronavirus: implications for virus origins and receptor binding, The Lancet. Published online Jan. 29, 2020. https://doi.org/10.1016/ S0140-6736(20)30251-8.
Sheahan, T. P. et al. Broad-spectrum antiviral GS-5734 inhibits both epidemic and zoonotic coronaviruses. Sci Transl Med 9, doi:10.1126/scitranslmed.aal3653 (2017).
Sheahan, T. P. et al. An orally bioavailable broad-spectrum antiviral inhibits SARS-CoV-2 in human airway epithelial cell cultures and multiple coronaviruses in mice. Sci Transl Med 12, doi:10.1126/scitranslmed.abb5883 (2020).
Sheahan, T. P. et al. Comparative therapeutic efficacy of remdesivir and combination lopinavir, ritonavir, and interferon beta against MERS-CoV. Nat Commun 11, 222,
doi:10.1038/s41467-019-13940-6 (2020).
Wang, D. et al. Clinical Characteristics of 138 Hospitalized Patients With 2019 Novel Coronavirus-Infected Pneumonia in Wuhan, China. JAMA, doi:10.1001/jama.2020.1585 (2020).
Kotenko, S. V. et al. IFN-lambdas mediate antiviral protection through a distinct class II cytokine receptor complex. Nat Immunol 4, 69-77, doi:10.1038/ni875 (2003).
Sheppard, P. et al. IL-28, IL-29 and their class II cytokine receptor IL-28R. Nat Immunol 4, 63-68, doi:10.1038/ni873 (2003).
Andreakos, E. & Tsiodras, S. COVID-19: lambda interferon against viral load and hyperinflammation. EMBO Mol Med, doi:10.15252/emmm.202012465 (2020).
Li, L. et al. IFN-lambda preferably inhibits PEDV infection of porcine intestinal epithelial cells compared with IFN-alpha. Antiviral Res 140, 76-82, doi:10.1016/j.antiviral.2017.01.012 (2017).
Mordstein, M. et al. Lambda interferon renders epithelial cells of the respiratory and gastrointestinal tracts resistant to viral infections. J Virol 84, 5670-5677, doi:10.1128/JVI.00272-10 (2010).
Beigel JH, Tomashek KM, Dodd LE, et al. Remdesivir for the Treatment of Covid-19 - Final Report. N Engl J Med. October 2020;doi:10.1056/NEJMoa2007764
Horby P, Lim WS, Emberson JR, et al. Dexamethasone in Hospitalized Patients with Covid-19
Preliminary Report. N Engl J Med. July 2020;doi:10.1056/NEJMoa2021436
Aoki FY, Macleod MD, Paggiaro P, et al. Early administration of oral oseltamivir increases the benefits of influenza treatment. J Antimicrob Chemother. January 2003;51(1):123-9. doi:10.1093/jac/dkg007
Carrat F, Duval X, Tubach F, et al. Effect of oseltamivir, zanamivir or oseltamivir-zanamivir combination treatments on transmission of influenza in households. Antivir Ther. 2012;17(6):1085-90. doi:10.3851/IMP2128
Park A, Iwasaki A. Type I and Type III Interferons - Induction, Signaling, Evasion, and Application to Combat COVID-19. Cell Host Microbe. 06 2020;27(6):870-878. doi:10.1016/j.chom.2020.05.008
Prokunina-Olsson L, Alphonse N, Dickenson RE, et al. COVID-19 and emerging viral infections: The case for interferon lambda. J Exp Med. 05 2020;217(5)doi:10.1084/jem.20200653
Davidson S, McCabe TM, Crotta S, et al. IFNλ is a potent anti-influenza therapeutic without the inflammatory side effects of IFNα treatment. EMBO Mol Med. 09 2016;8(9):1099-112. doi:10.15252/emmm.201606413
Dinnon KH, Leist SR, Schäfer A, et al. A mouse-adapted model of SARS-CoV-2 to test COVID-19 countermeasures. Nature. August 2020;doi:10.1038/s41586-020-2708-8
Vanderheiden A, Ralfs P, Chirkova T, et al. Type I and Type III Interferons Restrict SARS-CoV-2 Infection of Human Airway Epithelial Cultures. J Virol. 09 2020;94(19)doi:10.1128/JVI.00985-20
Muir AJ, Arora S, Everson G, et al. A randomized phase 2b study of peginterferon lambda-1a for the treatment of chronic HCV infection. J Hepatol. December 2014;61(6):1238-46. doi:10.1016/j.jhep.2014.07.022
Chan HLY, Ahn SH, Chang TT, et al. Peginterferon lambda for the treatment of HBeAg-positive chronic hepatitis B: A randomized phase 2b study (LIRA-B). J Hepatol. May 2016;64(5):1011-1019. doi:10.1016/j.jhep.2015.12.018
Zheng S, Fan J, Yu F, et al. Viral load dynamics and disease severity in patients infected with SARS-CoV-2 in Zhejiang province, China, January-March 2020: retrospective cohort study. BMJ. 04 2020;369:m1443. doi:10.1136/bmj.ml443
Pujadas E, Chaudhry F, McBride R, et al. SARS-CoV-2 viral load predicts COVID-19 mortality. Lancet Respir Med. 09 2020;8(9):e70. doi:10.1016/S2213-2600(20)30354-4
Bullard J, Dust K, Funk D, et al. Predicting infectious SARS-CoV-2 from diagnostic samples. Clin Infect Dis. May 2020;doi:10.1093/cid/ciaa638
La Scola B, Le Bideau M, Andreani J, et al. Viral RNA load as determined by cell culture as a management tool for discharge of SARS-CoV-2 patients from infectious disease wards. Eur J Clin Microbiol Infect Dis. June 2020;39(6):1059-1061. doi:10.1007/s10096-020-03913-9
“Regeneron’s REGN-COV2 Antibody Cocktail Reduced Viral Levels and Improved Symptoms in Non-Hospitalized COVID-19 Patients,” Press Release, Regeneron Pharmaceuticals, Inc., Sept. 29, 2020, available at https://investor.regeneron.com/news-releases/news-release-details/regenerons-regn-cov2-antibody-cocktail-reduced-viral-levels-and.
Scohy A, Anantharajah A, Bodeus M, Kabamba-Mukadi B, Verroken A, Rodriguez-Villalobos H. Low performance of rapid antigen detection test as frontline testing for COVID-19 diagnosis. J Clin Virol. 08 2020;129:104455. doi:10.1016/j.jcv.2020.104455
Vidali S, Morosetti D, Cossu E, et al. D-dimer as an indicator of prognosis in SARS-CoV-2 infection: a systematic review. ERJ Open Res. April 2020;6(2). doi:10.1183/23120541.00260-2020
Zhou F, Yu T, Du R, et al. Clinical course and risk factors for mortality of adult inpatients with COVID-19 in Wuhan, China: a retrospective cohort study. Lancet. 03 2020;395(10229):1054-1062. doi:10.1016/S0140-6736(20)30566-3
Naymagon L, Zubizarreta N, Feld J, et al. Admission D-dimer levels, D-dimer trends, and outcomes in COVID-19. Thromb Res. August 2020;196:99-105. doi:10.1016/j.thromres.2020.08.032
Bastard P, Rosen LB, Zhang Q, et al. Auto-antibodies against type I IFNs in patients with life-threatening COVID-19. Science. September 2020. doi:10.1126/science.abd4585
Zhang Q, Bastard P, Liu Z, et al. Inborn errors of type I IFN immunity in patients with life-threatening COVID-19. Science. September 2020. doi:10.1126/science.abd4570
Hadjadj J, Yatim N, Barnabei L, et al. Impaired type I interferon activity and inflammatory responses in severe COVID-19 patients. Science. 08 2020;369(6504):718-724. doi:10.1126/science.abc6027
Vermeiren C, Marchand-Senécal X, Sheldrake E, et al. Comparison of Copan ESwab and FLOQSwab for COVID-19 Diagnosis: Working around a Supply Shortage. J Clin Microbiol. 05 2020;58(6). doi:10.1128/JCM.00669-20
Corman VM, Landt O, Kaiser M, et al. Detection of 2019 novel coronavirus (2019-nCoV) by real-time RT-PCR. Euro Surveill. 01 2020;25(3). doi:10.2807/1560-7917. ES.2020.25.3.2000045
Cao B, Wang Y, Wen D, et al. A Trial of Lopinavir-Ritonavir in Adults Hospitalized with Severe Covid-19. N Engl J Med. March 2020. doi:10.1056/NEJMoa2001282
Hermant P, Michiels T. Interferon-λ in the context of viral infections: production, response and therapeutic implications. J Innate Immun. 2014;6(5):563-74. doi:10.1159/000360084
Syedbasha M, Egli A. Interferon Lambda: Modulating Immunity in Infectious Diseases. Front Immunol. 2017;8:119. doi:10.3389/fimmu.2017.00119
Loutfy MR, Blatt LM, Siminovitch KA, et al. Interferon alfacon-1 plus corticosteroids in severe acute respiratory syndrome: a preliminary study. JAMA. December 2003;290(24):3222-8. doi:10.1001/jama.290.24.3222
Etzion O, Hamid S, Lurie Y, et al. End of study results from LIMT HDV study: 36% durable virologic response at 24 weeks post-treatment with pegylated interferon lambda monotherapy in patients with chronic hepatitis delta virus infection. presented at: International Liver Congress 2019; 2019; Vienna, Austria.
Crotta S, Davidson S, MahlakoivT, et al. Type I and type III interferons drive redundant amplification loops to induce a transcriptional signature in influenza-infected airway epithelia. PLoS Pathog. 2013;9(11):e1003773. doi:10.1371/journal.ppat.1003773
Jin X, Lian JS, Hu JH, et al. Epidemiological, clinical and virological characteristics of 74 cases of coronavirus-infected disease 2019 (COVID-19) with gastrointestinal symptoms. Gut. March 2020. doi:10.1136/gutjnl-2020-320926
Zhang W, Du RH, Li B, et al. Molecular and serological investigation of 2019-nCoV infected patients: implication of multiple shedding routes. Emerg Microbes Infect. 2020;9(1):386-389. doi:10.1080/22221751.2020.1729071
Feld J, C K, Biondi M, et al. Peginterferon-lambda for the treatment of COVID-19 in outpatients: A phase 2, placebo-controlled randomized trial. Lancet Respiratory Medicine. 2020.
P J, Andrews J, Bonilla H, et al. Peginterferon Lambda-1a for treatment of outpatients with uncomplicated COVID-19: a randomized placebo-controlled trial. MedRxiv. 2020
Guedj J, Dahari H, Pohl RT, Ferenci P, Perelson AS. Understanding silibinin’s modes of action against HCV using viral kinetic modeling. J Hepatol. May 2012;56(5):1019-24. doi:10.1016/j.jhep.2011.12.012
Guedj J, Dahari H, Rong L, et al. Modeling shows that the NS5A inhibitor daclatasvir has two modes of action and yields a shorter estimate of the hepatitis C virus half-life. Proc Natl Acad Sci USA. March 2013;110(10):3991-6. doi:10.1073/pnas.1203110110
Larios OE, Coleman BL, Drews SJ, et al. Self-collected mid-turbinate swabs for the detection of respiratory viruses in adults with acute respiratory illnesses. PLoS One. 2011;6(6):e21335. doi:10.1371/journal.pone.0021335
Ge D, Fellay J, Thompson AJ, et al. Genetic variation in IL28B predicts hepatitis C treatment-induced viral clearance. Nature. September 2009;461(7262):399-401. doi:10.1038/nature08309
Thomas DL, Thio CL, Martin MP, et al. Genetic variation in IL28B and spontaneous clearance of hepatitis C virus. Nature. October 2009;461(7265):798-801. doi:10.1038/nature08463
Prokunina-Olsson L, Muchmore B, Tang W, et al. A variant upstream of IFNL3 (IL28B) creating a new interferon gene IFNL4 is associated with impaired clearance of hepatitis C virus. Nat Genet. February 2013;45(2):164-71. doi:10.1038/ng.2521

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

What is claimed is:

1. A method of treating a coronavirus infection in a subject, the method comprising subcutaneously administering to the subject a therapeutically effective amount of pegylated interferon lambda-1a until one or more of:
a sustained reduction of coronavirus viral load is reached,
a decrease in coronavirus RNA to undetectable levels is reached,
a decrease in a rate or an amount of viral shedding is reached, or
an improvement in the subject’s symptoms is reached.

2. The method of claim 1, wherein the pegylated interferon lambda-1a is administered for at least 1 week, 2 weeks, 3 weeks, 4 weeks, from 1-12 weeks, from 2-12 weeks, or from between 3 weeks and 24 weeks.

3. The method of claim 1 or 2, wherein the pegylated interferon lambda-1a is administered at a dose of 180 micrograms once a week, 90 micrograms twice per week, 80 micrograms twice per week, or 180 micrograms per week.

4. The method of claim 1 or 2, wherein the pegylated interferon lambda-1a is administered at a dose of 120 micrograms once a week, 60 micrograms twice per week, 70 micrograms twice per week, or 120 micrograms per week.

5. The method of claim 1 or 2, wherein the method comprises administering (i) 160 - 180 micrograms pegylated interferon lambda-1a per week for a first treatment period, and then 150 - 170 micrograms per week for a second treatment period; or (ii) 180 micrograms per week for a first treatment period, and then between 170 - 120 micrograms per week for a second treatment period, wherein doses administered for each of (i) and (ii) may be divided into more than one dose per week.

6. The method of claim 1 or 2, wherein the method comprises administering the pegylated interferon lambda-1a at a dose of 120 micrograms per week for a first treatment period, and then at a dose of 80 micrograms per week for a second treatment period; or at a dose of 180 - 120 micrograms per week for a first treatment period and then at a dose of 120 - 80 micrograms per week for a second treatment period, wherein the doses may be divided into more than one dose per week.

7. The method of claim 5, wherein the first treatment period is longer than the second treatment period, or the second treatment period is longer than the first treatment period, or first treatment period and the second treatment period are the same length of time.

8. The method of claim 5, wherein the first treatment period has a duration of at least 1 week, at least 2 weeks, at least 6 weeks, or at least 8 weeks.

9. The method of claim 1, wherein treatment results in a reduction of coronavirus viral load in the subject of at least 2.0 log₁₀ coronavirus RNA IU/mL serum.

10. The method of claim 1, wherein treatment results in an improvement in the subject’s symptoms.

11. The method of claim 1, wherein the improvement in a subject’s symptoms include a reduction in fever, feeling less tired, a decrease in coughs, less or no shortness of breath, decreased feeling of aches and pains, and less or no diarrhea.

12. The method of claim 1, wherein treatment results in a coronavirus viral load that is below the level of detection.

13. The method of claim 1, wherein the method further comprises administering to the subject an antiviral.

14. The method of claim 13, wherein the antiviral comprises one or more of remdesivir, chloroquine, tenofovir, entecavir, protease inhibitors (lopinavir/ritonavir).

15. The method of claim 1, wherein prior to treatment, the subject has a baseline viral load of up to about 10⁴ coronavirus RNA copies per mL sample.

16. The method of claim 1, wherein a durable virologic response (DVR) is seen in the subject after administration.

17. The method of claim 1, where the subject has one or more of the following symptoms: pneumonia, fever, cough, shortness of breath, and muscle ache.

18. The method of claim 1, wherein the coronavirus is a zoonotic virus.

19. The method of claim 1, wherein the pegylated interferon lambda-1a is administered during an early phase of the coronavirus infection, and wherein the method shortens the duration of the coronavirus infection and prevents development of respiratory complications.

20. The method of claim 19, wherein the early phase of the coronavirus infection comprises one or more of: days 1-10 after initial viral load is determined, prior to experiencing respiratory symptoms that require hospitalization; a period when the subject is experiencing mild to moderate respiratory symptoms; a period when the subject is asymptomatic; or a period when the subject displays mild symptoms of respiratory infection with no respiratory distress.

21. The method of claim 20, wherein the mild symptoms of respiratory infection with no respiratory distress comprises a temperature <39.0° C., respiratory rate < 25, O2% Sat > 95% in room air or with supplemental oxygen through nasal cannula, or P/F ratio > 150.

22. The method of claim 1, wherein the subject has not demonstrated one or more of the following abnormal laboratory test in the 12 months prior to administration: platelet count <90,000 cells/mm3; white blood cell (WBC) count <3,000 cells/mm3; absolute neutrophil count (ANC) <1,500 cells/mm3; hemoglobin <11 g/dL for women and <12 g/dL for men; estimated creatinine clearance (CrCI) < 50 mL/min by Cockroft-Gault formulation; ALT and/or ALT levels > 10 times the upper limit of normal; bilirubin level ≥ 2.5 mg/dL unless due to Gilbert’s syndrome; serum albumin level <3.5 g/dL; or international normalized ratio (INR) ≥1.5 (except patients maintained on anticoagulant medications).

23. The method of claim 1, wherein the rate or amount of viral shedding is determined by RT-PCR negativity or a measurement of a reduced amount of virus.

24. The method of claim 1, wherein an improvement in symptoms is determined by clinical improvement O2 status.

25. The method of claim 1, wherein the subject is a mild, non-hospitalized subject; a mild to moderate, non-hospitalized subject; a mild to moderate, hospitalized subject; a mild to moderate, hospitalized and requiring supportive O2 subject; or an exposed subject with no symptoms.

26. The method of claim 1, wherein the pegylated interferon lambda-1a is administered at a dose of 120 or 180 mcg weekly.

27. The method of claim 1, wherein the subject is a mild to moderate, hospitalized subject, and wherein the pegylated interferon lambda-1a is administered as one or two doses of 120 or 180 mcg weekly.

28. The method of claim 1, wherein RT-PCR is used to test for viral load at days 7 and 14 of treatment, and wherein the subject exhibits lower viral loads at days 7 and 14 than a patient with similar disease status at initiation of treatment that received only standard supportive care.

29. The method of claim 1, wherein the subject is a mild to moderate subject, and wherein the subject exhibits a decreased rate or amount of viral shedding.

30. The method of claim 1, wherein the subject is a mild to moderate, hospitalized subject requiring supportive O2, and wherein the subject demonstrates clinical improvement in oxygen status (ordinal scale) as compared to a patient with similar disease status at initiation of treatment that only received standard supportive care.

31. The method of claim 30, wherein the subject is administered two doses of interferon lambda one week apart.

32. The method of claim 1, wherein the subject has mild to moderate disease and is non-hospitalized or hospitalized, wherein the pegylated interferon lambda-1a is administered at a dose of 120 or 180 mcg twice weekly, and wherein the subject exhibits a lower rate of viral shedding as measured by RT-PCR negativity at Day 7 and/or Day 14 of treatment.

33. A method of preventing or reducing incidence of infection of a subject with SARS-CoV-2, the method comprising administering to the subject interferon lambda by subcutaneous injection in a dose of 120 or 180 mcg weekly or biweekly, wherein the subject is RT-PCR negative at Day 14 after a first dose of interferon lambda.

34. The method of claim 33, wherein the subject has a lower RT-PCR level of SARS-CoV-2 than a subject receiving standard supportive care.

35. A method of preventing or reducing incidence of a SARS-CoV-2 infection in a subject exposed to SARS-CoV-2, the method comprising administering to the subject 180 mcg of interferon lambda as a subcutaneous injection, wherein the subject exhibits a lower viral load at day 7 after the injection than a subject with similar disease status at initiation of treatment receiving standard supportive care.

36. The method of claim 35, wherein the subject exhibits a lower conversion rate to infection than a patient with similar disease status at initiation of treatment that was not administered interferon lambda.

37. The method of claim 35 or 36, wherein the subject has had exposure to SARS-CoV-2 with no confirmed infection.

38. A method of treating a subject having a SARS-CoV-2 infection or having been exposed to SARS-CoV-2, the method comprising administering to the subject interferon lambda at a dose of 180 mcg, wherein the subject has one or more of the following, as compared to a control:
a reduced duration of viral shedding of SARS-CoV-2 virus,
a reduction in the duration of symptoms, or
a reduction in the rate of hospitalization between Day 1 and Day 28 of treatment.

39. The method of claim 38, wherein the interferon lambda is administered subcutaneously.

40. The method of claim 38 or 39, wherein the interferon lambda is interferon lambda-1a.

41. The method of claim 38, wherein the interferon lambda is pegylated interferon lambda.

42. The method of claim 38, wherein the rate of hospitalization includes visits to an emergency room.

43. The method of claim 1, wherein the subject has a viral load equal to or greater than 6 log₁₀ copies/mL.

44. The method of claim 1, wherein the subject has a viral load of from about 6 log₁₀ IU/mL to about 11 log₁₀ IU/mL.

45. A method of treating a coronavirus infection in a subject, the method comprising subcutaneously administering to the subject from 120 mcg to 180 mcg of interferon lambda, wherein the subject has a viral load greater than or equal to 10⁶ SARS-CoV-2 RNA copies/mL or greater than or equal to 6 log₁₀ IU/mL.

46. The method of claim 45, wherein the interferon lambda is administered at a dose of 120 mcg or 180 mcg, and wherein the subject exhibits a lower rate of viral shedding as measured by viral load negativity at Day 7, Day 14 and/or Day 28 of treatment as compared to at the initiation of treatment.

47. The method of claim 45 or claim 46, wherein the subject has a viral load of from about 6 log₁₀ IU/mL to about 11 log₁₀ IU/mL.

48. The method of claim 1 or claim 45, wherein the time to shedding cessation is faster in a seropositive subject relative to a seronegative subject at baseline.

49. The method of claim 45, wherein the subject has a greater decline in SARS-CoV-2 RNA viral load decline from baseline at Day 5 of treatment, as compared to a control.

50. The method of claim 46, wherein the subject is about 4.1-fold or 95% more likely to clear virus by Day 7 of treatment, as compared to a control.

51. The method of claim 46, wherein the subject has a viral load greater than or equal to 6 log₁₀ IU/mL, and wherein the subject is viral negative by Day 7 of treatment.

52. The method of claim 46, wherein the subject clears the virus by Day 7 of treatment.

53. The method of claim 46, wherein the interferon lambda is pegylated interferon lambda-1a.
